(19) United States
(12) Patent Application Publication
(10) Pub. No.: US 2026/0306172 A1
(43) Pub. Date: Oct. 8, 2026

(54) HIGH-PRECISION MEDICAL TREATMENT SYSTEMS IN THREE OR HIGHER DIMENSIONS AND METHODS UTILIZING A SET OF PULSED LASERS

(71) Applicant: ULTRAFAST MEDICAL TECHNOLOGY (SHANGHAI) CO., LTD., Shanghai (CN)

(72) Inventors: Tongxin LI, Shanghai (CN); Tingxia LI, Shanghai (CN); Hongsheng JIN, Shanghai (CN)

(73) Assignee: ULTRAFAST MEDICAL TECHNOLOGY (SHANGHAI) CO., LTD., Shanghai (CN)

(21) Appl. No.: 19/206,891

(22) Filed: May 13, 2025

Related U.S. Application Data
(63) Continuation of application No. PCT/CN2025/087423, filed on Apr. 7, 2025.

(51) Int. Cl. A61B 18/20; A61B 18/00; A61B 34/10; A61B 90/00; G16H 40/63

(52) U.S. Cl. CPC A61B 18/20; A61B 34/10; A61B 90/37; G16H 40/63; A61B 2018/00184; A61B 2018/0047; A61B 2018/00601; A61B 2018/00642; A61B 2018/00702; A61B 2018/00761; A61B 2018/20353; A61B 2018/20355; A61B 2018/20359; A61B 2018/205547; A61B 2034/107; A61B 2090/373

(57) ABSTRACT

The present disclosure describes systems, methods, and non-transitory computer-readable media designed for high-precision medical treatments in 3D and higher dimensions, utilizing a set of pulsed lasers. In one embodiment, a medical treatment system includes a laser source that is designed to generate a set of pulsed lasers. This system also features a scanning module that focuses the laser pulses into a set of spots, which are then directed onto a treatment area. A scanning control module is responsible for managing the movement of the scanning module in space, enabling the focused laser spots to cover the treatment area in a specific scan pattern. A controller coordinates the operation of the laser source, scanning module, and scanning control module, ensuring synchronized function throughout the treatment process.

### CROSS REFERENCE TO RELATED APPLICATIONS

This application is a continuation of International Application No. PCT/CN2025/087423, filed on Apr. 7, 2025, entitled “HIGH-PRECISION MEDICAL TREATMENT SYSTEMS IN THREE OR HIGHER DIMENSIONS AND METHODS UTILIZING A SET OF PULSED LASERS,” which is hereby incorporated by reference in its entirety.

### TECHNICAL FIELD

This disclosure pertains broadly to semiconductor technology, and more specifically, to systems and techniques employed in medical treatments that utilize lasers.

### BACKGROUND

Lasers possess properties such as coherence, high monochromaticity, and the capability to generate high power, making them suitable for various medical applications, including diagnostics, therapy, and surgery in fields such as ophthalmology, dentistry, oncology, cosmetics, and dermatology. Current approaches typically involve either directing a single, high-power, large-area light spot at the treatment site or utilizing multiple split laser beams across the surface of the skin. However, the intense energy of each pulse can potentially harm surrounding tissues due to thermal effects, leading to significant side effects like pigmentation, scarring, and visible blood streaks. Conversely, these existing methods often suffer from suboptimal treatment efficiency because of the relatively low laser power density.

### SUMMARY

One aspect of the present disclosure provides a system for medical treatment, comprising: a laser source configured to generate a series of pulsed lasers; a scanning module configured to provide a series of focused laser spots based on the series of pulsed lasers; a scanning control module configured to control spatial movement of the scanning module to scan the series of focused laser spots on a treatment area to form a scan pattern; and a controller configured to control the laser source, the scanning module, and the scanning control module.

In some embodiments, the spatial movement of the scanning module comprises at least three linear movements along three linear directions perpendicular with each other.

In some embodiments, the spatial movement of the scanning module comprises at least two linear movements along two linear directions perpendicular with each other, and at least one rotating movement along an angular direction.

In some embodiments, the spatial movement of the scanning module comprises at least a linear movement along a linear direction, a rotating movement along an angular direction, and a radial movement along a radial direction.

In some embodiments, the spatial movement of the scanning module further comprises a roll movement, a yaw movement, and a pitch movement.

In some embodiments, the system further comprises: a stage configured to hold a patient including the treatment area; wherein the controller is further configured to control spatial movement of the stage.

In some embodiments, the spatial movement of the stage comprises at least three linear movements along three linear directions perpendicular with each other.

In some embodiments, the spatial movement of the stage comprises at least two linear movements along two linear directions perpendicular with each other, and at least one rotating movement along an angular direction.

In some embodiments, the spatial movement of the stage comprises at least a linear movement along a linear direction, a rotating movement along an angular direction, and a radial movement along a radial direction.

In some embodiments, the spatial movement of the stage further comprises a roll movement, a yaw movement, and a pitch movement.

In some embodiments, a duration of each of the pulsed lasers is in a range between 1 ns and 1,000 ns.

In some embodiments, a duration of each of the pulsed lasers is in a range between 1 ps and 1,000 ps.

In some embodiments, a duration of each of the pulsed lasers is in a range between 1 fs and 1,000 fs.

In some embodiments, each of the pulsed lasers includes a series of bursts, and each of the focused laser spot is formed by the series of bursts.

In some embodiments, the scanning module is further configured to focus each of the pulsed lasers such that a dimension of each of the focused laser spots is in a range between 0.1 μm and 500 μm.

In some embodiments, the scanning module comprises a scanning galvanometer and at least one laser objective lens.

In some embodiments, the controller is further configured to: receive detection data indicative of a treatment area of the patient; and provide a control instruction indicative of the scan pattern based on the detection data.

In some embodiments, the system further comprises: an image module configured to, prior to the medical treatment, detect the treatment area; and provide the detection data to the controller.

In some embodiments, the detection data is indicative of at least one of a texture, a shape, a color, and a defect of the treatment area.

In some embodiments, the image module is further configured to, during the medical treatment: detect the scan pattern; and provide updated detection data to the controller based on the detection of the scan pattern.

In some embodiments, the scanning control module is further configured to control spatial movement of the scanning module to move the series of focused laser spots on the patient to form an updated scan pattern based on the updated detection data.

In some embodiments, the controller is further configured to adjust a size and/or a shape of the scan pattern to form the updated scan pattern.

In some embodiments, the controller is further configured to adjust a path of the movement of the focused laser spots to form the updated scan pattern.

In some embodiments, the system further comprises: the image module includes a hyperspectral imaging camera configured to capture images of the treatment area across multiple spectral bands, providing diagnostic data.

In some embodiments, the controller is further configured to: process the detection data using a trained AI model to automatically identify and classify a condition type of the treatment area.

In some embodiments, the controller is further configured to adjust powers applied by the focused laser spots during the medical treatment.

In some embodiments, to adjust the powers applied by the focused laser spots, the controller is further coupled to the laser source and configured to control the laser source to adjust at least one of a power of the laser source and a number of bursts in each of the pulsed lasers.

In some embodiments, to adjust the powers applied by the focused laser spots, the controller is further configured to control the scanning module to adjust at least one of a speed and a repetition of the movement of the focused laser spots.

In some embodiments, the system further comprises: an optical fiber configured to transmit the series of pulsed lasers from the laser source to the scanning module.

In some embodiments, the optical fiber is a hollow-core fiber or a gain fiber.

In some embodiments, the system further comprises: a distance measuring module configured to measuring a distance between the scanning module and locations of the treatment area.

In some embodiments, the controller is further configured to: generate a 3D data model of treatment area based on outputs from the image module and the distance measuring module; and control the scanning module and the scanning control module to adjust at least some of the focused laser spots to form at different depths under a skin of the patient based on the 3D data model of treatment area.

In some embodiments, the series of pulsed lasers include picosecond lasers or femtosecond lasers; and the treatment area includes curved human tissues.

In some embodiments, the series of pulsed lasers include picosecond lasers or femtosecond lasers; and the treatment area includes deep human tissues.

In some embodiments, the series of pulsed lasers include picosecond lasers or femtosecond lasers; and scanning the series of focused laser spots on the treatment area includes interacting the series of focused laser spots with human tissues at different depths.

In some embodiments, the series of pulsed lasers include picosecond lasers or femtosecond lasers; and scanning the series of focused laser spots on the treatment area includes generating thermal effects, cutting effects, or perforation effects.

In some embodiments, the series of pulsed lasers include picosecond lasers or femtosecond lasers; and

scanning the series of focused laser spots on the treatment area includes locating the series of focused laser spots on target harmful cells and tissues on or within organs, enabling non-invasive destruction of undesirable biological structures.

Another aspect of the present disclosure provides a method for medical treatment, comprising: detecting, by an image module, a treatment area of a patient; generating, by a laser source, a series of pulsed lasers; providing, by a scanning module, a series of focused laser spots based on the series of pulsed lasers; and moving, by a scanning control module, the series of focused laser spots on the patient to form a scan pattern based on the detected treatment area of the patient.

Another aspect of the present disclosure provides a non-transitory computer-readable medium containing computer-executable instructions that, when executed by a processor, cause the processor to perform a method for medical treatment, the method comprising: detecting, by an image module, a treatment area of a patient; generating, by a laser source, a series of pulsed lasers; providing, by a scanning module, a series of focused laser spots based on the series of pulsed lasers; and moving, by a scanning control module, the series of focused laser spots on the patient to form a scan pattern based on the detected treatment area of the patient.

### BRIEF DESCRIPTION OF THE DRAWINGS

The drawings provided herein, which are an integral part of this specification, depict various embodiments of the present disclosure. Along with the accompanying description, they help clarify the underlying principles of the disclosure and provide sufficient information for a person skilled in the relevant field to implement and utilize the disclosure.

FIG. 1 shows a schematic representation of an exemplary system designed for medical treatment utilizing focused laser spots, in accordance with certain embodiments of the present disclosure.

FIG. 2 presents a schematic diagram of a representative controller, as described in some embodiments of the present disclosure.

FIG. 3 depicts exemplary pulsed lasers emitting a set of bursts, as outlined in some embodiments of the present disclosure.

FIGS. 4A-4C demonstrate different exemplary scan patterns created by moving a set of focused laser spots over the treatment areas of a patient, in accordance with various embodiments of the present disclosure.

FIG. 5 shows a cross-sectional view of a typical treatment area where a set of focused laser spots are applied, as described in some embodiments of the present disclosure.

FIG. 6A illustrates a representative application of focused laser spots in aesthetic medicine, according to certain embodiments of the present disclosure.

FIG. 6B depicts a representative dental application of focused laser spots, as outlined in certain embodiments of the present disclosure.

FIG. 6C illustrates an exemplary use of focused laser spots in orthopedics, according to some embodiments of the present disclosure.

FIG. 6D shows an exemplary oncology application of focused laser spots, in accordance with specific embodiments of the present disclosure.

FIGS. 7A to 7C depict an example of a treatment device featuring a movement platform, as described in certain embodiments of the present disclosure.

FIGS. 8A to 8D demonstrate representative movements of a treatment apparatus, in accordance with specific embodiments of the present disclosure.

FIG. 9 shows an example of a scanning module, as outlined in certain embodiments of the present disclosure.

FIG. 10A presents an example of a scanning area that covers a treatment area, based on particular embodiments of the present disclosure.

FIG. 10B illustrates an example of a scanning path within a scanning area, according to some embodiments of the present disclosure.

FIGS. 11A to 11C showcase an example of a treatment apparatus with a movement platform, as per some embodiments of the present disclosure.

FIGS. 12A to 12F highlight typical movements of a treatment device, in line with certain embodiments of the present disclosure.

FIGS. 13A to 13B provide an example of a treatment apparatus equipped with a movement platform, according to certain embodiments of the present disclosure.

FIG. 14A shows an example of a scanning area that encompasses a treatment area, in accordance with certain embodiments of the present disclosure.

FIG. 14B depicts an example of a scanning path within a scanning area, as described in some embodiments of the present disclosure.

FIGS. 15A and 15B illustrate an example of a treatment device featuring a movement platform, according to some embodiments of the present disclosure.

FIG. 16A demonstrates an exemplary scanning area covering a treatment zone, based on specific embodiments of the present disclosure.

FIG. 16B shows a representative scanning path within a scanning area, as per certain embodiments of the present disclosure.

FIG. 17 presents a flowchart outlining a method for medical treatment utilizing focused laser spots, according to certain embodiments.

FIGS. 18A-18F provide examples of movements of a stage carrying a patient in the medical treatment system, according to certain embodiments of the present disclosure.

FIG. 19 depicts an example of a laser head in the medical treatment system, according to certain embodiments of the present disclosure.

FIGS. 20A-20I provide examples of scanning multiple galvanometer scanning areas of a large treatment area by the medical treatment system, according to certain embodiments of the present disclosure.

FIGS. 21A-21F provide examples of a laser forcing method for different treatment areas at different depths of skin using the medical treatment system, according to certain embodiments of the present disclosure.

FIG. 22 depicts an example of a laser-based method for treating dermal pigmentation, according to certain embodiments of the present disclosure.

FIGS. 23A to 23B showcase examples of a treatment apparatus with a robotic arm, as per some embodiments of the present disclosure.

FIGS. 24A to 24B showcase examples of a treatment apparatus with a robotic arm, as per some embodiments of the present disclosure.

FIG. 25 presents a flowchart outlining a method for medical treatment utilizing robotic arm, according to certain embodiments.

Embodiments of the present disclosure will be described with reference to the accompanying drawings.

### DETAILED DESCRIPTION

While particular configurations and arrangements are described for illustrative purposes, it should be understood that these are merely examples. A person skilled in the relevant field will recognize that alternative configurations and arrangements can be utilized without deviating from the essence or scope of the present disclosure. It will be evident to those with expertise in the field that the present disclosure is also applicable to a wide range of other potential uses.

It is to be understood that references in the specification to “one embodiment,” “an embodiment,” “an example embodiment,” “some embodiments,” etc., mean that the embodiment described may include a particular feature, structure, or characteristic, but every embodiment may not necessarily contain the particular feature, structure, or characteristic. Additionally, such terms do not always refer to the same embodiment. When a specific feature, structure, or characteristic is described in relation to one embodiment, it will be apparent to a person skilled in the relevant field that this feature, structure, or characteristic could also apply to other embodiments, whether or not explicitly mentioned.

Terminology, in general, can be understood in part from the context in which it is used. For instance, the term “one or more” may refer to a singular feature, structure, or characteristic, or it could refer to combinations of features, structures, or characteristics, depending on the context. Similarly, terms like “a,” “an,” or “the” may be interpreted as singular or plural, again depending on the context. The phrase “based on” should not be taken to imply an exclusive set of factors, but rather may accommodate additional factors that are not necessarily described, depending on the context.

In certain existing laser-based medical systems, unfocused laser spots of relatively large size (e.g., greater than 1 mm in diameter) are applied directly to blood vessels, teeth, bones, tumors, or other tissues or organs. Although the energy per pulse is high in these systems, the overall energy density remains relatively low due to the large size of the laser spots. The high energy of a single pulsed laser can cause irreparable damage to human tissue due to its peak power. In addition, the longer duration of a single pulsed laser (e.g., greater than 100 ps) can lead to thermal damage to nearby tissues because of heat transfer through thermal conduction. In some existing systems, a single pulse laser can be divided into multiple smaller laser spots arranged in a matrix pattern. However, this division also disperses the energy across each individual laser spot, which in turn reduces the overall energy density.

Various embodiments of the present disclosure provide medical treatment systems that use focused laser spots delivered by low-duration pulsed lasers. By reducing the spot size and the pulse duration, the energy density can be significantly increased, thus enhancing the efficiency and effectiveness of the treatment. Moreover, the shorter duration of each pulse (e.g., less than 50 ps) helps to prevent thermal damage to surrounding tissues, as it is shorter than the time required for heat to conduct to adjacent tissues. Additionally, by utilizing a set of focused laser spots over time instead of an array of laser spots simultaneously, the energy density of each laser spot is preserved. This approach allows for greater control over the treatment area and parameters, which can be easily adjusted by modifying the scan patterns created by the series of focused laser spots.

The disclosed medical treatment system employs a laser with an ultrashort pulse width, reaching femtosecond durations, and operates at relatively low power, resulting in minimal damage to the body. This ensures a safe and efficient treatment with no recovery time, reduces side effects, and minimizes pain, all of which significantly enhance the user experience. The system also integrates a hyperspectral camera and leverages artificial intelligence (AI) to accurately identify the treatment area and dynamically adjust the laser parameters. Using precise femtosecond laser pulses, the system gently and effectively targets both the surface and dermis of the skin, helping to reduce fine lines, lighten pigmentation and redness, and improve uneven skin tone, dullness, and hyperpigmentation, thus restoring the skin's natural radiance.

In addition, the system includes a 3D or 6D motion platform with multiple linear and/or rotating motor modules. The laser objective lens can move freely in 3D space based on commands from the controller, allowing the laser to focus on planes at varying heights. Compared to conventional galvanometers and φ-θ field lenses, the combination of the 3D/6D motion platform and objective lens significantly expands the scanning range, reduces the size of the field lens, enhances scanning efficiency, and lowers overall costs.

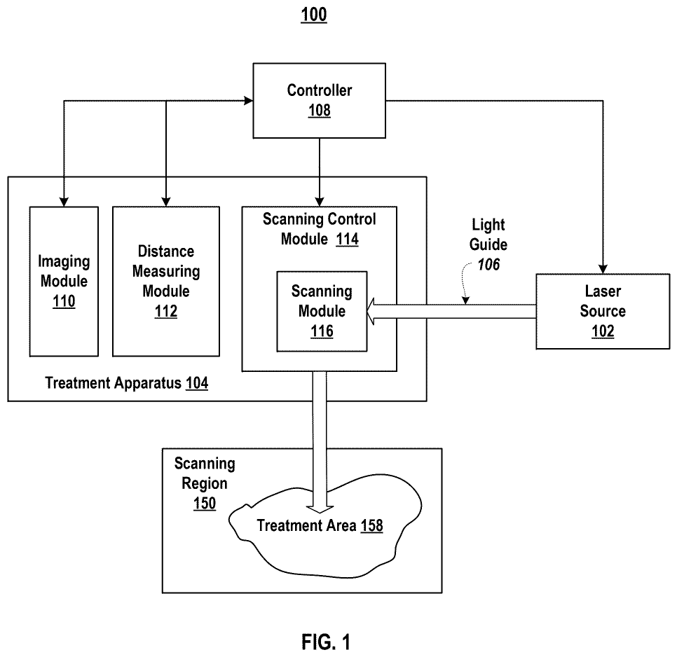

FIG. 1 presents a schematic diagram of an exemplary system 100 for medical treatment utilizing focused laser spots, in accordance with certain embodiments of the present disclosure. System 100 includes a laser source 102, a scanning control module 104, and a controller 108. Laser source 102 can be any appropriate type of laser, including but not limited to fiber lasers, solid-state lasers, gas lasers, liquid lasers, and semiconductor lasers. Laser source 102 is configured to generate a set of pulsed lasers at various wavelengths, such as 532 nm, 600-1,000 nm, 1,064 nm, 1,550 nm, 2,940 nm, among others. In some embodiments, the power output of laser source 102 may be dynamically controlled by controller 108 during the treatment process.

Laser source 102 may operate as either a continuous laser or a pulsed laser, depending on the application. In certain embodiments, the duration of each laser pulse is in the nanosecond (ns) range. Specifically, the pulse duration may range from approximately 1 ns to 1000 ns, including values such as 1 ns, 2 ns, 3 ns, up to 1,000 ns, or any subset of these values, including ranges defined by any two of these points. In other embodiments, the duration of each pulsed laser is in the picosecond (ps) range. For instance, the duration of each pulsed laser may range from approximately 1 ps to 1000 ps, with possible values including 1 ps, 2 ps, 3 ps, up to 1000 ps, or any value within or between these ranges. In further embodiments, the duration of each pulsed laser may be in the femtosecond (fs) range. Specifically, the duration can range from about 50 fs to 1000 fs, including values such as 50 fs, 60 fs, 70 fs, 80 fs, 90 fs, 100 fs, 200 fs, 300 fs, 400 fs, 500 fs, 600 fs, 700 fs, 800 fs, 900 fs, 1,000 fs, or any subset or range defined by these values. In some embodiments, the pulse duration is significantly shorter than the time required for electron energy to transfer to the material lattice. This allows each pulsed laser to complete its effect before the thermal energy released through gasification can cause thermal damage to surrounding tissues via thermal conduction.

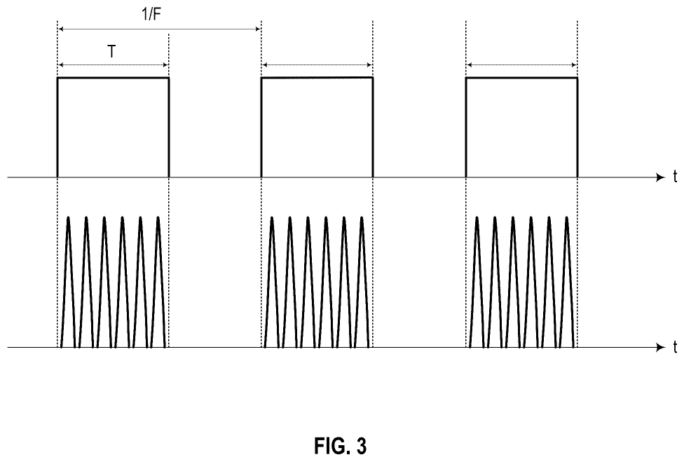

Each pulsed laser can be delivered as a single pulse or as a set of bursts. FIG. 3 illustrates an example of pulsed lasers operating in a burst mode, according to certain embodiments of the present disclosure. The pulsed lasers are generated by the laser source at a frequency F, with the pulse width of each laser pulse denoted as T. In burst mode, multiple bursts, denoted as N, can be generated within the same pulse width T, where N is an integer greater than 1, typically ranging from 2 to 100. In some embodiments, the burst frequency is on the order of 1 nanosecond (ns), while the overall frequency F of the laser pulses is on the order of 1 microsecond (μs). For example, the pulse frequency F may range from about 1 Hz to about 50 MHz. As a result, the laser energy is first accumulated within each burst (on the scale of ns), and then further accumulated over the pulse duration (on the scale of μs), allowing for a very high energy density without increasing the peak energy. Thus, each focused laser spot may consist of 1 to NNN bursts.

Referring to FIG. 1, in some embodiments, the pulsed lasers generated by laser source 102 may have a single wavelength or multiple wavelengths, such as two or three distinct wavelengths. Pulses with different wavelengths can be generated either separately, simultaneously, or in an alternating manner. In some embodiments, the wavelength of the pulsed lasers generated by laser source 102 ranges from 100 nm to 1,100 nm, with examples including 520 nm, 532 nm, 775 nm, 1,040 nm, 1,964 nm, 1,550 nm, and 2,940 nm. The output frequency of laser source 102 may range from 20 kHz to 1,000 kHz in some embodiments. In certain embodiments, the average output power of laser source 102 is between 0 W and 100 W, or between 1 W and 50 W. These values for the pulsed lasers and laser source 102 are provided for illustrative purposes and are not limiting.

Scanning control module 104 is optically coupled to laser source 102 via light guide 106 and includes an imaging module, a distance measuring module 112, and a scanning control module 114. The scanning control module 104 is configured to generate a set of focused laser spots within a scanning region 150, based on the pulsed lasers generated by laser source 102. The treatment area 158 of a patient is positioned within the scanning region 150. In some embodiments, scanning control module 104 is operatively connected to controller 108, receiving control signals and instructions from the controller.

In some embodiments, scanning control module 114 may include a scanning module 116. Additionally, scanning control module 114 may further incorporate a three-dimensional (3D) or six-dimensional (6D) movement module, which includes multiple linear motor modules and/or angular motor modules. These components enable the scanning module 116 to move in 3D or 6D space. Based on control signals from controller 108, scanning control module 114 can direct scanning module 116 to move freely within 3D space, adjusting its position and/or direction. This allows the laser beam to focus on a targeted location within the treatment area 158, such as on planes at different heights. In this way, scanning module 116 can scan the pulsed lasers within a specified scan angle and scan rate, as determined by the scanning control module 114, toward a designated location in the three-dimensional scanning region 150.

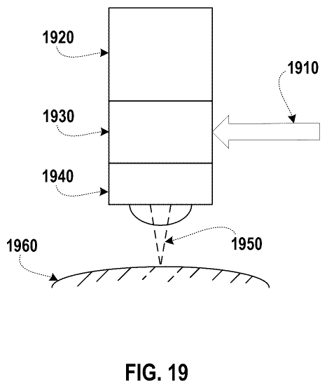

In some embodiments, as shown in FIG. 19, scanning module 116 includes a scanning galvanometer 1930, a laser objective lens 1940, and/or a polarizer (not shown). The scanning module 116 may also include any other suitable scanning mirrors and refractive optics. Furthermore, scanning module 116 is configured to receive the pulsed lasers 1910 from laser source 102 through light guide 106, focusing each pulse to form a set of focused laser spots 1950 on the treatment area 1960. In some embodiments, the dimension of each focused laser spot 1950 ranges from 1 micrometer (μm) to 500 μm, with possible values such as 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, or any range bounded by any of these values. The shape of each focused laser spot 1950 may vary and can include, for example, round, rectangular, square, irregular, or other suitable forms. In some embodiments, each focused laser spot 1950 has a generally round shape, with a diameter ranging from 1 μm to 300 μm. It is understood that the dimensions of the series of focused laser spots 1950 may be either consistent or variable across the spots. By focusing the pulsed laser beams 1910 into individual focused laser spots 1950, the energy density can be greatly increased.

Referring back to FIG. 1, the laser system 100 may employ a variety of optical transmission methods, including but not limited to light guide arms, multiple-mirror combinations, and fiber optic transmission. In some embodiments, light guide 106 is configured to transmit the laser from laser source 102 to scanning module 116 utilizing any of these suitable optical transmission methods, such as light guide arm transmission, mirror combination transmission, or fiber optic transmission. In certain embodiments, light guide 106 may specifically include a light guide arm. To optimize performance and accommodate the varying motion patterns of scanning control module 114 and/or scanning module 116 in 3D space, the shape and length of the light guide arm can be tailored to maximize the high-precision characteristics of the arm. This ensures precise light transmission and accurate positioning of the laser beam. During the dynamic operation of scanning module 116, the light guide arm effectively directs the laser output from laser source 102 along the optical path with minimal power loss, guiding the laser accurately to the center of the scanning galvanometer. This ensures precise energy transfer and efficient utilization of the laser. In some embodiments, light guide 106 may incorporate a combination of multiple mirrors. These mirrors are designed for high reflectivity at specific laser wavelengths and are arranged in a particular geometric configuration on scanning control module 114. As scanning control module 114 and/or scanning module 116 move, the mirror arrangement efficiently transmits the laser output from laser source 102 along the optical path to the center of the galvanometer with very little power loss. In some embodiments, light guide 106 may include one or more fibers, such as hollow-core fibers and/or gain fibers.

System 100 may also include a stage designed to support the patient and position the treatment area 158 within scanning region 150. The stage can include an operating table, a hospital bed, or any other suitable platform for supporting the patient during medical treatment. Additionally, the stage may be configured, under the control of controller 108, to adjust the patient's position during the treatment process. The stage can move the patient's entire body or specific body parts, such as the head, teeth, arms, legs, or other areas as needed.

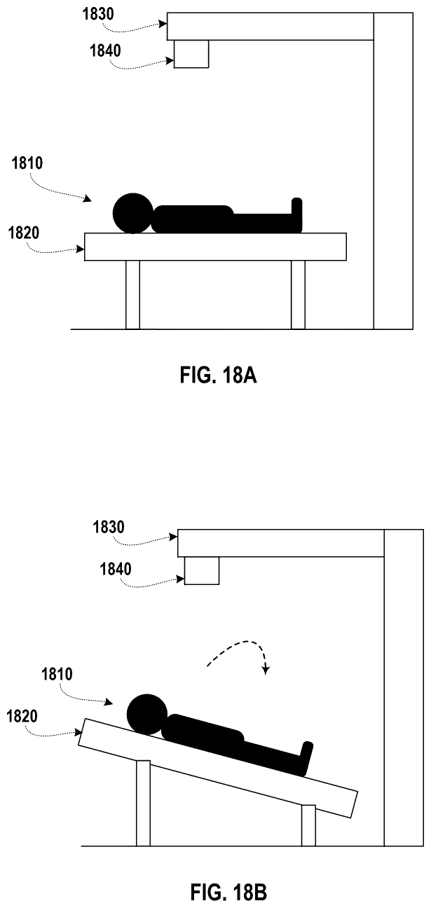

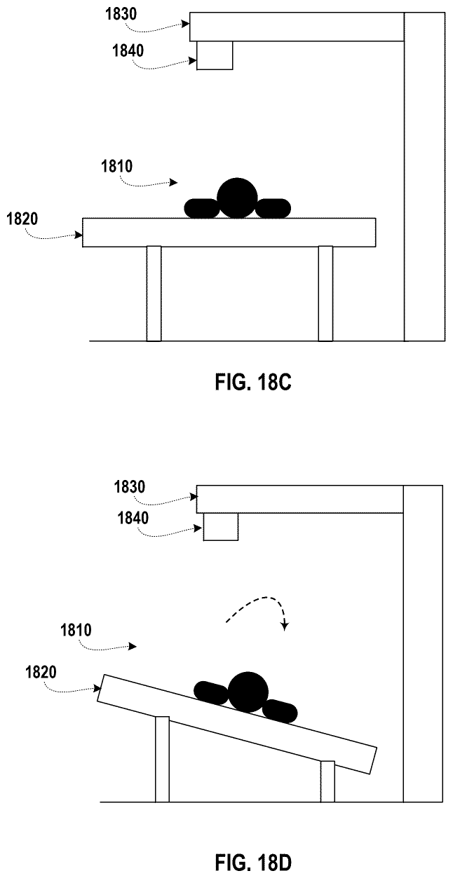

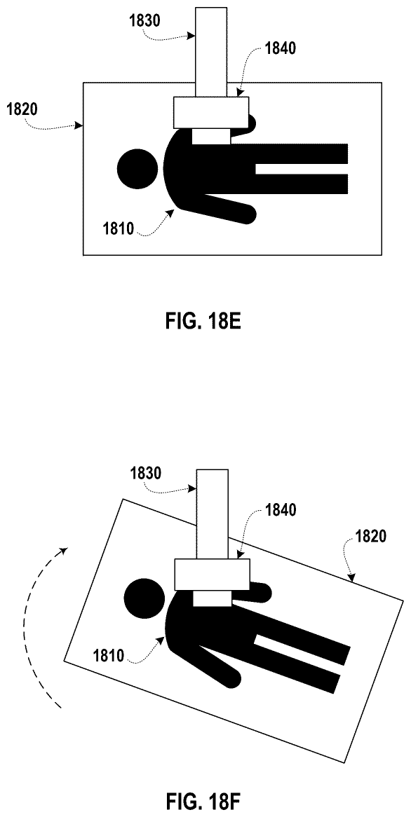

Referring to FIGS. 18A-18F, a stage 1820 carrying a patient 1810 can be positioned under a laser head 1840 mounted on a laser motion platform 1830. In some embodiments, the stage 1820 may be capable of precise 3D/6D movement to facilitate accurate positioning of the treatment area 158 within the scanning region 150. For example, stage 1820 can move along the X, Y, and Z axes to align treatment area 158 with the laser beam path delivered by laser head 1840. In addition to linear movement, stage 1820 may offer rotational adjustments and/or tilting adjustments of the patient 1810 with respect to different axes to expose a specific angle of the patient 1810 to optimize the orientation of the treatment area within the scanning region 150. For example, as shown in FIGS. 18A-18B, stage 1820 can tilt the patient 1810 along a Y-axis (front-back tilt) to adjust the patient's angle to facilitate access to anterior or posterior regions, such as chest, back, top of head, soles of feet, etc. As another example, as shown in FIGS. 18C-18D, stage 1820 can tilt along the X-axis (left-right tilt) to help expose lateral areas of the patient 1810, such as the side of the head or torso. As yet another example, as shown in FIGS. 18E-18F, stage 1820 can rotate along the Z-axis (rotational tilt) provide a spiral orientation, ideal for aligning curved anatomical structures, such as the arms, legs, or spinal column. These multidirectional rotating and tilting capabilities enhance the system's adaptability, ensuring precise laser delivery to complex or hard-to-reach treatment areas.

Referring back to FIG. 1, imaging module 110 may be configured to detect the treatment area 158, either before or during the treatment, and send detection data to controller 108. Imaging module 110 can include, but is not limited to, a camera, thermal imaging device, X-ray machine, ultrasound machine, or other suitable medical imaging equipment. Imaging module 110 can be integrated into scanning control module 104 or operate as a standalone device, separate from the scanning control module. For example, imaging module 110 may be a dedicated medical imaging device that captures medical images of treatment area 158 and sends those images, or any data derived from them, to controller 108. Additionally, the detection of treatment area 158 can be performed utilizing a variety of modalities, such as images, videos, sounds, or text. Beyond capturing initial detection data of the treatment area, imaging module 110 may also perform continuous monitoring during the treatment or upon request by the operator, allowing for real-time tracking of treatment progress, such as detecting the scan pattern applied to the patient.

In some embodiments, imaging module 110 may include a specialized hyperspectral camera that integrates high-diffraction-efficiency transmission gratings for spectral separation, high-sensitivity area-array cameras, built-in scanning sensors, and one or more auxiliary cameras. This configuration eliminates the need for external push-broom imaging mechanisms and complex focusing adjustments that are typically required by traditional hyperspectral cameras. Imaging module 110 can provide real-time transmission of hyperspectral inspection images to an HD display, enabling detailed observation of treatment area 158 and precise targeting during laser operation. In some embodiments, imaging module 110 may feature the following key capabilities: (1) Hyperspectral Imaging: Coverage from 400 nm to 1,000 nm with a spectral resolution of better than 2.5 nm, offering up to 240 spectral channels. (2) High Resolution: High pixel density for detailed image capture and clarity. (3) Durability and Reliability: Built with robust, low-maintenance construction suitable for various working conditions. (4) Programmability: Supports automated image capture, processing, and analysis. (5) Flexibility: Customizable for optimal image capture, analysis, and processing tailored to specific application needs.

In some embodiments, distance measuring module 112 includes an infrared distance sensor that uses triangulation to scan treatment area 158. This enables the creation of a 3D data model of the treatment area through computer processing, ensuring precise positioning in coordination with the 3D/6D motion of scanning module 116. In these embodiments, the infrared sensor emits an infrared beam at a specific angle, which reflects off the treatment area 158 and returns to the sensor. By measuring the position of the reflected light on the sensor (e.g., a CCD or CMOS sensor), a triangle is formed with the infrared distance sensor at the apex. This allows the distance between the sensor and the point in treatment area 158 to be calculated utilizing geometric relationships. In some embodiments, distance measuring module 112 may feature the following key capabilities: (1) High Accuracy: Provides millimeter-level precision at close range, making it ideal for applications that require precise measurements. (2) Strong Adaptability: Capable of accurately measuring various target types, including reflective, dark, bright, and absorptive surfaces. (3) Non-Contact Measurement: Eliminates errors and potential damage associated with physical contact, unlike traditional contact-based measurement devices.

In other embodiments, distance measuring module 112 may incorporate a spectral confocal displacement sensor 1920, as shown in FIG. 19. Using the dispersion-based confocal principle, spectral confocal displacement sensor 1920 enables high-precision distance measurements without being affected by optical aberrations. Unlike traditional sensors, the spectral confocal displacement sensor 1920 maintains accuracy even when the treatment area is tilted at steep angles. The spectral confocal displacement sensor 1920 performs exceptionally well on reflective, transparent, or semi-transparent surfaces and offers a strong tolerance to angular variations. This means high-precision measurements can be achieved without the need for strict angle adjustments, and the spectral confocal displacement sensor 1920 is less sensitive to vibrations or slight movements of the treatment area during measurement. Therefore, by including a spectral confocal displacement sensor 1920, distance measuring module 112 can deliver precise and stable measurements across a wide variety of surface types, even in demanding conditions.

In certain embodiments, controller 108 is connected to laser source 102, scanning control module 104, and/or the stage 1820, enabling it to manage their operations through control signals and commands. The controller 108 is designed to direct scanning control module 114, which moves scanning module 116 to reposition the focused laser spots within scanning region 150, thus creating the desired scan pattern. When scanning module 116 directs the focused pulsed lasers toward treatment area 158, any relative movement between the stage 1820 and scanning module 116 will result in the shifting of the focused laser spots across treatment area 158, thereby generating a scan pattern according to this relative motion. By adjusting the relative movement between the stage 1820 and scanning module 116, such as speed and direction, controller 108 can manipulate the scan pattern's size and shape within scanning region 150.

In some configurations, controller 108 can also control the positioning of the focused laser spots at various depths within the skin of treatment area 158. For instance, the laser focus can be fine-tuned to target specific depths within a range of 0 to 20 mm beneath the skin's surface. With this functionality, the laser can precisely target tissues or blood vessels at different depths within the specified range, enabling effective therapeutic treatments. This precise depth control facilitates selective laser treatment of subcutaneous tissues, ensuring that surrounding areas remain unaffected, which is particularly advantageous for dermatological and vascular applications.

In certain embodiments, controller 108 can further adjust the scan pattern by modifying the pulsed laser parameters generated by laser source 102, thus fine-tuning the laser's operation. In some cases, the scan pattern may be manually set by a medical professional-such as a physician or technician-who determines it based on clinical expertise, treatment protocols, and/or the patient's current condition. Alternatively, in some embodiments, controller 108 may automatically or semi-automatically generate the scan pattern based on input from imaging module 110 and/or distance measuring module 112.

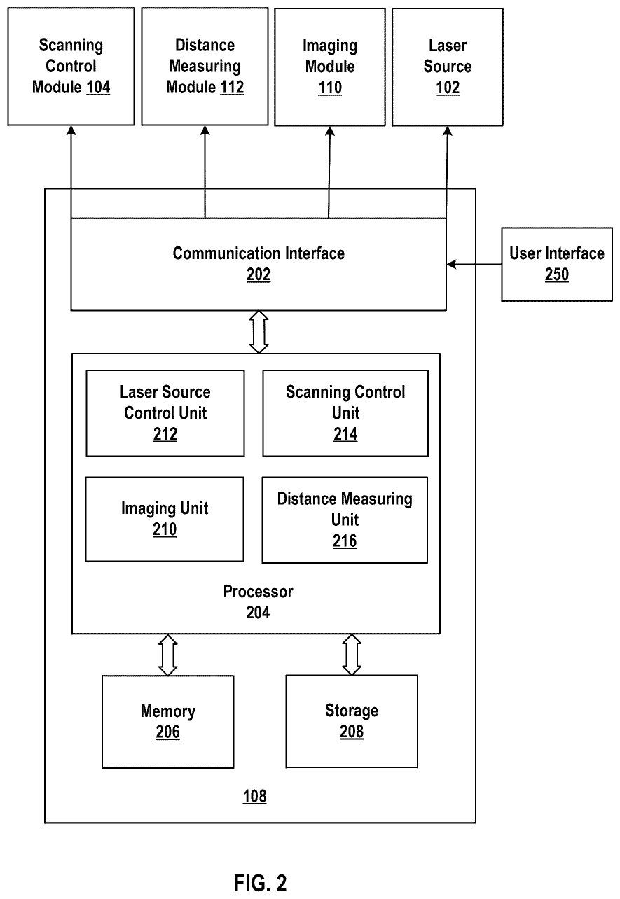

FIG. 2 provides a schematic illustration of the controller 108, according to certain embodiments of the present disclosure. Controller 108 is responsible for controlling the operations of laser source 102, imaging module 110, distance measuring module 112, scanning control module 114, and/or the stage 1820. For example, it generates, adjusts, and positions a set of focused laser spots on the treatment area 158 to create a scan pattern based on the detected treatment area 158 of the patient. In some embodiments, controller 108 receives data from imaging module 110 and distance measuring module 112 that indicates the characteristics of the treatment area 158, and generates control signals based on this data to adjust the scan pattern. These control signals are then sent to laser source 102, scanning control module 114, and/or the stage 1820.

As shown in FIG. 2, controller 108 can include various components such as a communication interface 202, a processor 204, a memory 206, and storage 208. In some cases, controller 108 may integrate these components into a single device, such as an integrated circuit (IC) chip, implemented as either an application-specific integrated circuit (ASIC) or a field-programmable gate array (FPGA). Alternatively, these functions could be split across separate devices with dedicated roles. The components of controller 108 may be located together with the laser source 102 and/or scanning control module 104 as part of the overall system 100, or they could be positioned remotely, such as in a standalone computing device, in the cloud, or at another external location. The components of controller 108 can either be integrated into a single device or distributed across different locations, communicating with each other via a network (not shown). For instance, processor 204 may be located on-board laser source 102 and/or scanning control module 104, within a standalone computing device, in the cloud, or in any combination of these configurations.

The communication interface 202 facilitates data transmission between components such as laser source 102, imaging module 110, distance measuring module 112, scanning control module 114, and/or the stage 1820. This communication can occur through various means, including wired communication cables, Wireless Local Area Network (WLAN), Wide Area Network (WAN), radio frequency wireless networks, nationwide cellular networks, or local wireless networks (e.g., Bluetooth™ or Wi-Fi), or any other suitable communication methods. In some cases, communication interface 202 may include an integrated services digital network (ISDN) card, a cable modem, a satellite modem, or another type of modem for establishing data communication connections. Alternatively, communication interface 202 could be a local area network (LAN) card, enabling connectivity to a compatible LAN. Additionally, communication interface 202 can support wireless communication. In such a configuration, communication interface 202 is capable of sending and receiving electrical, electromagnetic, or optical signals that transmit digital data streams representing various types of information over a network. In certain embodiments, communication interface 202 can be connected to a user interface (UI) 250 for interaction with the system.

Processor 204 can be any suitable type of general-purpose or specialized processor, such as a microprocessor, digital signal processor (DSP), or microcontroller. Processor 204 may be configured as a dedicated module focused on controlling laser source 102, imaging module 110, distance measuring module 112, scanning control module 114, and/or the stage 1820. Alternatively, processor 204 could be configured as a shared processing unit that performs other tasks unrelated to the operation of the laser source 102, imaging module 110, distance measuring module 112, scanning control module 114, and/or the stage 1820.

As illustrated in FIG. 2, processor 204 may comprise multiple functional units, such as an imaging unit 210, a laser source control unit 212, a scanning control unit 214, a distance measuring unit 216, and a stage control unit (not shown), among others. These units (and their respective sub-units) can be hardware components (e.g., parts of an integrated circuit) within processor 204, specifically designed to manage the corresponding modules (e.g., modules 110-116 in FIG. 1) or execute program segments. The program can be stored on a computer-readable medium, and when executed by processor 204, it enables the performance of one or more functions. Although FIG. 2 shows units 210-216 all within one processor 204, it is contemplated that these units may be distributed among multiple processors located near or remotely with each other.

Imaging unit 210 is configured to determine the scan pattern formed by the series of focused laser spots, utilizing detection data provided by imaging module 110 from treatment area 158. In certain embodiments, imaging unit 210 receives detection data that reflects characteristics of treatment area 158 from imaging module 110. As previously discussed, the treatment area 158 may include various parts of the body depending on the medical treatment, such as a portion of a lesion, tumor, tooth, skin, blood vessel, or any other tissue or organ. The detection data may include information about the texture, shape, color, defects, or other relevant features of the treatment area 158 that are pertinent to the medical treatment being applied. For instance, imaging unit 210 may employ appropriate image processing algorithms to analyze the images of treatment area 158 and generate detection data from the analysis in the form of images, videos, sounds, texts, or metadata. In some embodiments, the image processing algorithms may include pattern recognition methods that use machine learning to automatically extract feature information from the treatment area, such as its texture, shape, color, and defects, based on pre-trained data.

Distance measuring unit 214 is designed to create 3D data models of treatment area 158, utilizing the output from distance measuring module 112. In certain embodiments, the distances between the scanning module 114 and the laser focal point in treatment area 158 can be calculated utilizing a triangulation method, based on the data obtained from the CCD or CMOS sensor of distance measuring module 112.

Additionally, processor 204 can determine the scan pattern to be formed by the series of focused laser spots in treatment area 158, utilizing both the detection data from imaging module 110 and the 3D data model of treatment area 158 generated by distance measuring unit 214. The scan pattern for treatment area 158 can be defined by the series of focused laser spots. More specifically, processor 204 can determine both the size and shape of the scan pattern, as well as the parameters that define how the series of focused laser spots will form this pattern with the desired characteristics. For example, these parameters include, but are not limited to, the path, speed, and repetition of the laser spots' movement, the size of the spots, the spacing between adjacent spots, the density and repetitiveness of the spots, the depth of focus of the spots beneath the skin, the frequency of the laser spots, and any other factors that influence the final size and shape of the scan pattern or the energy delivered to the patient during the treatment.

In addition to determining the initial scan pattern dynamically based on the characteristics of the treatment area 158, processor 204 can also adaptively modify the scan pattern in real time throughout the course of the medical treatment. Throughout the treatment, detection data may be updated continuously or upon request and provided to imaging unit 210, while the 3D data model of treatment area 158 can be updated in real time or on demand by distance measuring unit 214. The updated detection data and 3D modeling of treatment area 158 provide information on the progress and condition of both the scan pattern and the treatment area during the medical procedure. Based on this real-time information, scanning control unit 214 can dynamically adjust the parameters related to the focused laser spots, reflecting the current status of the treatment area and scan pattern as indicated by the updated data. The subsequent focused laser spots can either continue to follow the parameters of the initial scan pattern or be modified to align with the updated parameters. For instance, the size or shape of the initial scan pattern may be altered to create a new scan pattern. Or the path along which the focused laser spots move may be adjusted to modify the scan pattern accordingly.

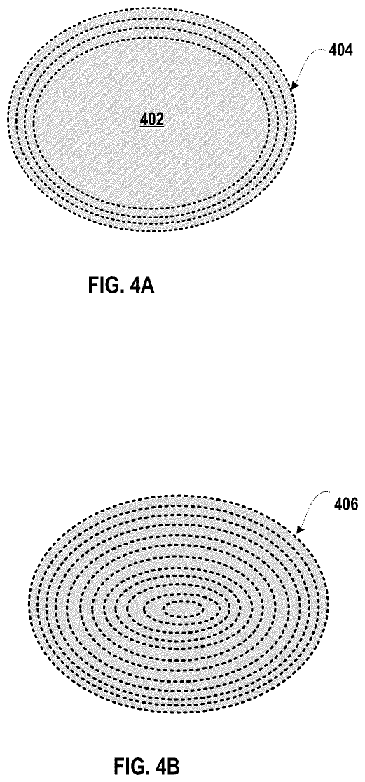

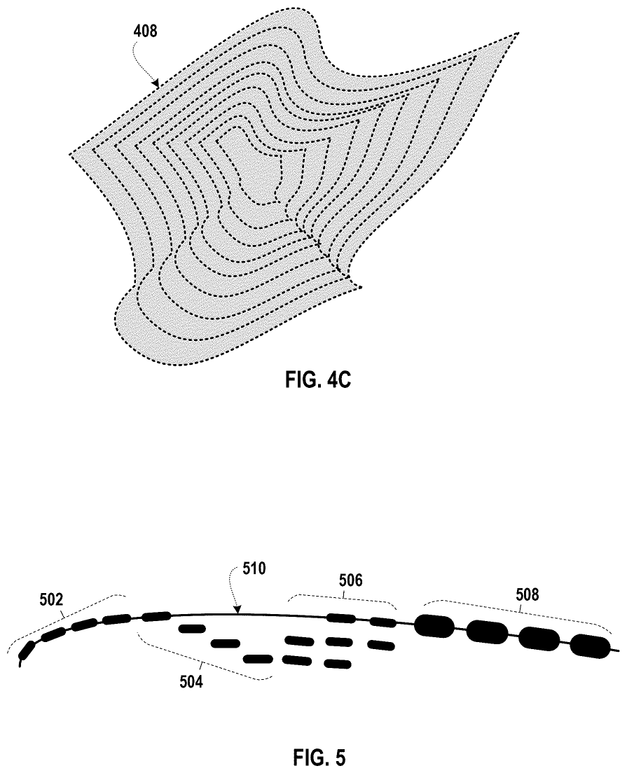

FIGS. 4A-4C illustrate a range of example scan patterns that are formed by moving a set of focused laser spots across various treatment areas of a patient, in line with different embodiments of this disclosure. As shown in FIG. 4A, treatment area 402 has an oval shape, and scan pattern 404 is derived based on the dimensions and shape of this treatment area, which may involve a set of elliptical patterns that change in size. Other factors for the focused laser spots are also defined, such as their arrangement (e.g., rectangular shape), size, density, and spacing. In FIG. 4B, an updated scan pattern 406 is generated during the treatment, reflecting changes based on the progression of the initial scan pattern 404 and/or the ongoing status of treatment area 402. The shape of the treatment area is not confined to any specific regular form, such as the oval shape depicted in FIGS. 4A and 4B. For instance, FIG. 4C illustrates a treatment area with an irregular shape, and in this case, scan pattern 408 is determined according to the contours and dimensions of the irregularly shaped treatment area.

FIG. 5 provides a cross-sectional view of a representative treatment area featuring a set of focused laser spots, in line with some embodiments of the present disclosure. This figure also highlights key parameters of the focused laser spots that influence the resulting scan pattern, such as depth and repetitiveness. In region 502 of the scan pattern, the series of focused laser spots are positioned at the skin's surface 510, with no repetition occurring at this location. As the treatment progresses, the depth of the laser spots gradually increases, penetrating deeper into the skin until reaching the deepest point at region 504. In region 506, multiple laser spots are formed repeatedly at the same position, but at varying depths. In region 508, the laser spots continue to be repeated at the same location, but with uniform depth, which may result in larger laser spots. In certain embodiments, the depth of penetration ranges from 0 to 20 mm beneath the skin's surface. It is important to note that the examples provided above, in relation to FIGS. 4A-4C and FIG. 5, are merely illustrative and demonstrate how Imaging unit 210 can determine the appropriate scan pattern based on the treatment area of a patient.

Furthermore, in the process of forming the scan pattern, the series of focused laser spots may either overlap substantially (e.g., more than 50% of the laser spots overlap), partially overlap (e.g., 50% or fewer of the spots overlap), or not overlap at all. The degree of overlap between adjacent focused laser spots can range from 0% to 100%, depending on the application. In certain embodiments, two adjacent laser spots may be positioned close to each other without overlapping, leaving a defined gap between them.

Referring to FIG. 2, laser source control unit 212 is responsible for sending control instructions to laser source 102, specifying either the initial or updated scan pattern. These instructions prompt laser source 102 to initialize and adjust the various parameters associated with the pulsed lasers, both before and during the medical treatment, based on the determined scan pattern. In some configurations, laser source control unit 212 can regulate the power output of laser source 102, which in turn influences the size, shape, and/or depth of the focused laser spots, as well as the intensity applied to the treatment area. Additionally, the number of pulses in each burst generated by laser source 102 may be controlled by laser source control unit 212, allowing adjustment of the laser spot's size, shape, and/or depth. Frequency adjustments made by laser source control unit 212 can also impact the frequency and offset of the focused laser spots during the treatment process.

Scanning control unit 214 is tasked with providing control instructions to scanning control module 104, defining the scan pattern to be used. These instructions enable scanning control module 104 to adjust parameters related to the series of pulsed laser spots, ensuring alignment with the initial or updated scan pattern both before and during the procedure. In certain embodiments, scanning control unit 214 influences scanning module 116 to modify the path, speed, and repetition of the focused laser spots, thereby affecting their overall power during treatment. Similarly, scanning control unit 214 can modify scanning module 114 to adjust the size and/or shape of the laser spots, which in turn affects their energy delivery to the patient.

Memory 206 and storage 208 are designed to hold various types of data necessary for processor 204 to function effectively. Both memory 206 and storage 208 may consist of volatile or non-volatile storage types, including magnetic, semiconductor, optical, and tape media, and may be either removable or non-removable. Examples include ROM, flash memory, dynamic RAM, or static RAM. These storage components are configured to store computer programs that processor 204 can execute, enabling it to control the operation of laser source 102, scanning control module 104, and the stage 1820 during medical treatments. For instance, memory 206 and/or storage 208 may be configured to store one or more programs that processor 204 can execute to manage the operations of laser source 102, scanning control module 104, and the stage 1820, as well as to process data to generate control instructions and other related signals.

Additionally, memory 206 and/or storage 208 can store various types of information and data that are used by processor 204 during operation. For example, detection data provided by imaging module 110, which reflects the treatment area of the patient, can be stored in memory 206 and/or storage 208. This data may either be permanently stored, removed periodically, or discarded immediately after each detection or scan, depending on the system's configuration.

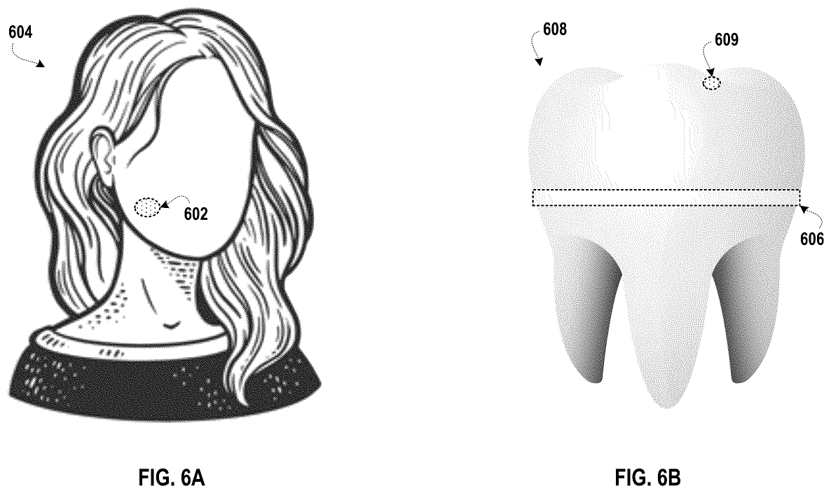

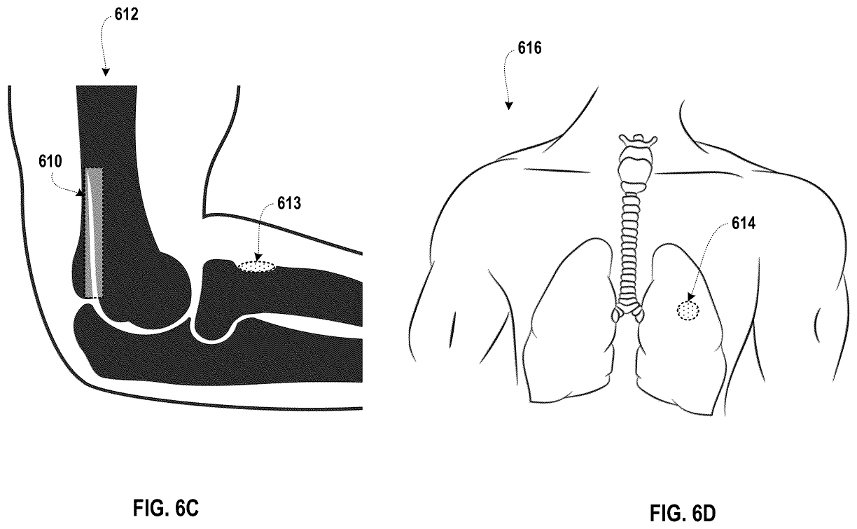

FIG. 6A illustrates an example of how focused laser spots can be applied in aesthetic medicine, according to some embodiments. As shown in FIG. 6A, scan pattern 602 is applied to a human face 604 or any other part of the body for aesthetic treatments, targeting areas such as scars, pigmented lesions, tattoos, skin irregularities, aging signs, skin laxity, wrinkles, moles, liver spots, excess fat, cellulite, unwanted hair, skin discoloration, and spider veins. Scan pattern 602 is determined based on the specific treatment area on the face or body and is formed by the movement of focused laser spots. The power, size, and/or shape of each laser spot can be finely controlled to optimize treatment for aesthetic applications.

FIG. 6B shows a dental application of focused laser spots, according to some embodiments. In FIG. 6B, scan pattern 606 is applied to a tooth 608 for purposes such as dental cleaning, plaque removal, or decay treatment. In some cases, a through hole 609 may be created in the tooth 608 by repeatedly applying a set of focused laser spots to a small region with high energy density, such as during dental surgery.

FIG. 6C illustrates the use of focused laser spots in orthopedic applications, according to some embodiments. As shown in FIG. 6C, scan pattern 610 can be directed through the skin to the surface of a bone 612 to remove bone tissue. For orthopedic surgery, a through hole 613 can be created in bone 612 by repeatedly applying a set of highly focused laser spots to a small area, with a very high energy density.

FIG. 6D demonstrates an oncology application of focused laser spots, according to some embodiments. As shown in FIG. 6D, scan pattern 614 is formed through the skin to target the surface of an organ 616, allowing for precise removal of a tumor from the organ. Scan pattern 614 is determined based on the tumor's location and is formed by moving a set of focused laser spots over the treatment area.

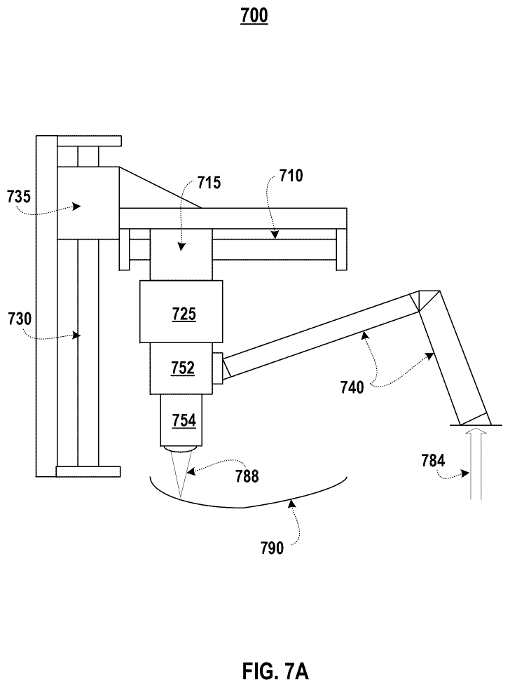

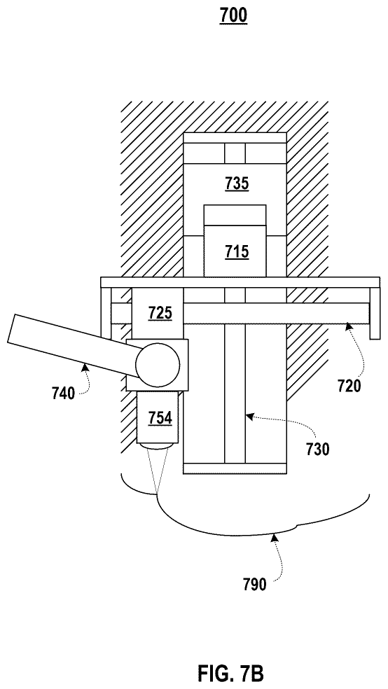

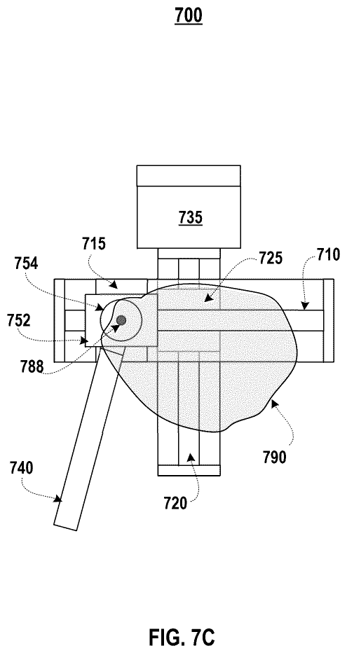

FIGS. 7A-7C illustrate an example of a treatment apparatus, according to some embodiments of the present disclosure. FIG. 7A shows treatment apparatus 700 in the X-Z plane, FIG. 7B shows the apparatus in the Y-Z plane, and FIG. 7C presents the apparatus in the X-Y plane. As depicted in FIGS. 7A-7C, treatment apparatus 700 includes a light guide 740, which is configured to transmit laser beam 784 from a laser source to a scanning module containing a scanning galvanometer 752 and a laser objective lens 754. In some embodiments, the length of light guide 740 can be tailored to meet specific treatment requirements, with the ability to adjust within a range that suits the conditions of the procedure. For example, the length may vary anywhere from 0.1 meters to 500 meters, depending on the needs of the treatment.

In other embodiments, scanning galvanometer 752 can be configured as either a 1D or 2D galvanometer, designed to adjust the angle of incidence of the laser beam through high-speed deflection. This capability enables efficient and precise scanning of the laser beam in both linear and planar directions, improving scan speed while reducing the active time of the laser. Scanning galvanometer 752 may include an optical scanning head, an electronic drive amplifier, and an optical mirror. The optical scanning head is equipped with a horizontal mirror mounted on a high-speed motor, allowing for rapid oscillation of the mirror. The electronic drive amplifier receives control signals from controller 108, which drives the optical scanning head to achieve the necessary deflections. The optical mirror is fixed onto the motor shaft and is engineered to reflect the laser beam with high reflectivity at specific wavelengths, thus minimizing power loss during the scanning process. The operating principle of scanning galvanometer 752 relies on the oscillation and reflection of the mirror. The laser beam is focused by an optical system and directed onto the galvanometer, where adjusting the frequency and amplitude of the 2D mirror's vibration enables precise control over the laser's scanning path across a plane. In some embodiments, the technical features of scanning galvanometer 752 can include the following: (1) High precision: This is achieved through a sophisticated control system and high-performance optics that ensure accurate laser beam positioning. (2) High speed: The system can achieve scanning rates of up to tens of thousands of points per second, enabling rapid and efficient operation. (3) Flexibility: The system offers adjustable scan paths and speeds, making it adaptable to a wide range of applications. (4) Stability: An advanced negative feedback loop design enhances system stability and reliability, minimizing potential errors and ensuring consistent performance.

In some embodiments, laser objective lens 754 is a specially designed near-infrared field lens that ensures a consistent focal point across the scanning plane during laser sweeping, achieving a flat-field effect. The lens coatings and optical design are optimized for the specific wavelength of the laser beam 784, and the materials used for the lens are selected to match the laser's output power. This ensures that the lens remains durable and unaffected by the high energy of the laser without risk of damage. After focusing through laser objective lens 754, the size of the focused laser spot 788 can be adjusted within a range from 1 μm to 500 μm, depending on the treatment requirements.

In some embodiments, the disclosed medical treatment system features a 3D/6D movement platform that supports multi-axis movement for precise control. This platform, driven by advanced motion control algorithms and a high-speed processor, enables real-time adjustments to ensure accuracy during treatment. The 3D/6D movement platform may comprise multiple linear motor modules that enable movement along different axes. The X and Y axis modules allow for precise horizontal movement, ensuring accurate positioning of the laser objective lens across the treatment area. The Z-axis motor module, which supports both the scanning module and the distance sensor module, enables vertical adjustments, controlling the distance between the laser objective lens and the treatment surface, ensuring both precise focus and accurate data collection.

As shown in FIGS. 7A-7C, the scanning module, which includes the scanning galvanometer 752 and laser objective lens 754, can be mounted on the scanning control module, which is powered by the 3D movement platform. In some implementations, the 3D movement platform consists of three linear motion portions: First linear motion portion (X-portion 715) along the X-axis 710. Second linear motion portion (Y-portion 725) along the Y-axis 720. Third linear motion portion (Z-portion 735) along the Z-axis 730. Each of these portions is independently driven by a linear motor module, allowing for precise movement along each respective axis. The independent control of these motion portions enables highly accurate positioning and movement of the laser system.

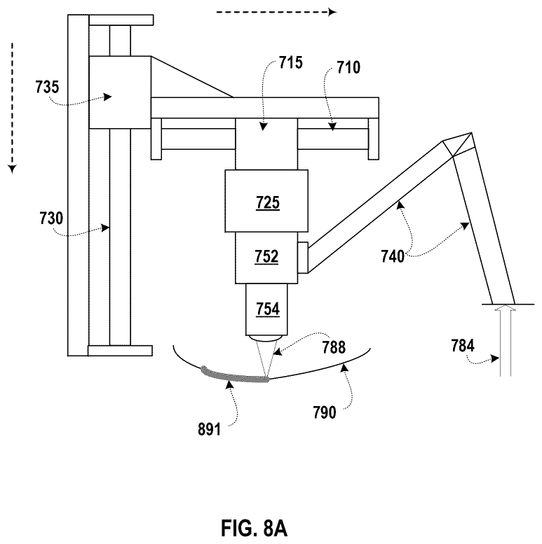

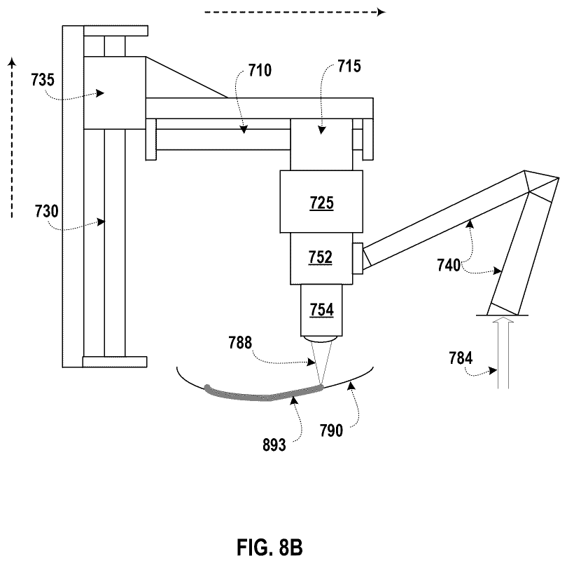

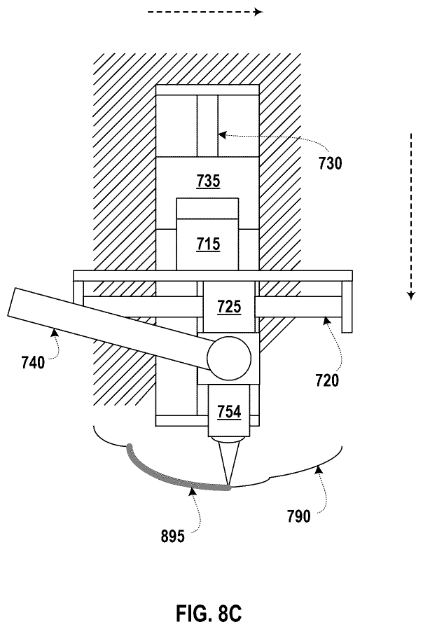

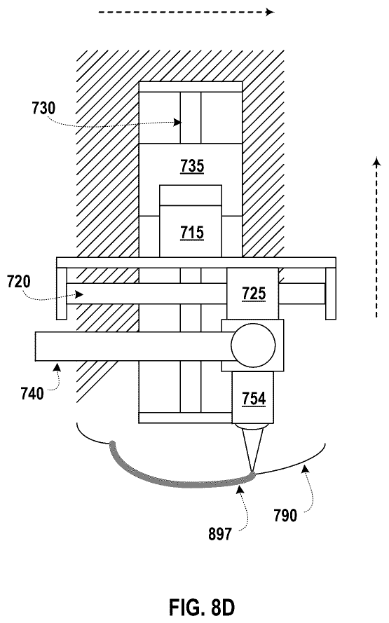

In some embodiments, as illustrated in FIG. 8A, the X-portion 715 can move at a constant or variable speed along the X-axis 710 (positive direction, +X), while the Z-portion 735 moves at a constant or variable speed along the Z-axis 730 (negative direction, −Z). This allows the laser beam 784 to cover portions of the treatment area 790 in a curved pattern (shown as curved line 891 in the X-Z plane). Similarly, as shown in FIG. 8B, the X-portion 715 can again move along the X-axis 710 (positive direction, +X), but this time the Z-portion 735 moves along the Z-axis 730 (positive direction, +Z). In this configuration, the laser beam 784 sweeps across portions of the treatment area 790 along another curved path (illustrated as curved line 893 in the X-Z plane). The laser beam 784 can cover portions of the treatment area 790 along a curved line 893 in the X-Z plane.

In some embodiments, in the Y-Z plane, as shown in FIG. 8C, the second linear motion portion 725 (Y-portion 725) moves at a constant or non-constant speed in the positive Y direction (+Y), while the third linear motion portion 735 (Z-portion 735) moves at a constant or non-constant speed in the negative Z direction (−Z). This motion allows the laser beam 784 to cover portions of the treatment area 790 along a curved line 895 in the Y-Z plane. Similarly, as shown in FIG. 8D, the second linear motion portion 725 (Y-portion 725) moves at a constant or non-constant speed in the positive Y direction (+Y), while the third linear motion portion 735 (Z-portion 735) moves at a constant or non-constant speed in the positive Z direction (+Z). In this configuration, the laser beam 784 can cover portions of the treatment area 790 along a curved line 897 in the Y-Z plane.

As previously discussed, the scanning galvanometer 752 can be either a 1D galvanometer or a 2D galvanometer. In some embodiments, as shown in FIG. 8, the laser beam 784 from light guide 740 reflects off the 2D scanning galvanometer 752 and passes through the laser objective lens 754, providing focused linear coverage on the treatment area projection (projection area 980) by controlling the mirror's rapid oscillation. This configuration of a 2D galvanometer combined with a field lens is effective for scanning flat surfaces but typically requires a large field lens to cover the entire treatment area. In other embodiments, by adding a 1D galvanometer to the XYZ movement platform, the X and Y axes define the laser's path as a fine line, while the 1D galvanometer enables broader, “thick-line” scanning. This combination accelerates the scanning rate by reducing the movement frequency of the X-Y motors and the laser activation time, which ultimately improves treatment efficiency. Table 1 below presents a comparison between the three different scanning setups. Based on the results of this comparison, the combination of a multi-axis movement platform with a 1D/2D/3D galvanometer and scan lens or objective lens setup proves to be highly effective in handling 3D skin contours. This configuration enhances the overall efficiency of the treatment process without requiring an increase in the size of the laser spot. It is noted that the objective lens can be reflective objectives or reflective telescope objectives.

TABLE 1

| | | | XYZ Platform + |
|---|---|---|---|
| | | | 1D/2D/3D |
| | | 2D | galvanometer + |
| | | galvanometer + | Scan lens/ |
| | XYZ Platform | field lens | Objective lens |
| Scanning Speed | Very slow | Fast | Relatively fast |
| Focus Spot Size | Small | Large | Small |
| Spot consistency | Good | Poor | Good |
| Focus Position | Accurate | Average | Accurate |
| Control | | | |
| Focus Depth | Support | Not support | Support |
| Control | | | |
| Treatment | Depending on | Depending on | Depending on |
| Format | XY platform | field lens size | XY platform |
| | travel | | travel |
| Structure Size | Depending on | Depending on | Depending on |
| | XY platform | field lens size | scan lens/ |
| | size | and focal length | objective lens |
| | | | size |
| Laser Power | Low | High | Low |
| Requirement | | | |

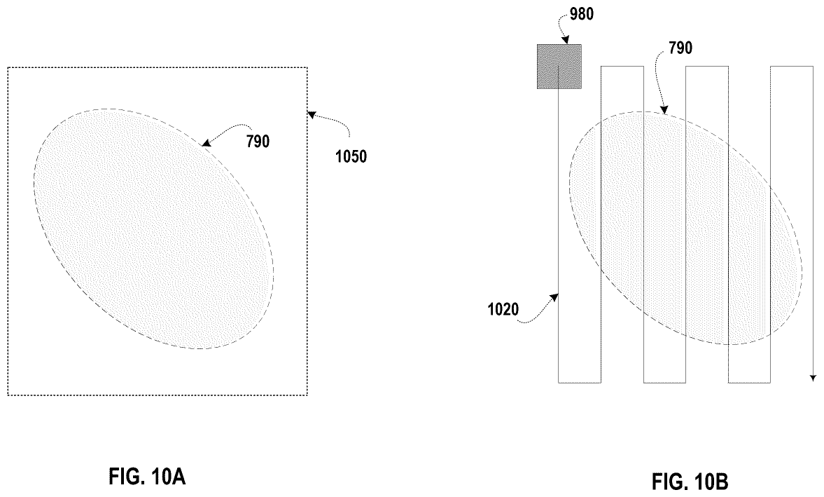

In some embodiments, within the X-Y plane, the combined movements of the first linear motion portion 715 (X-portion 715) and second linear motion portion 725 (Y-portion 725) along their respective X-direction and Y-direction enable the projection area 980 to follow a defined trajectory 1020, as illustrated in FIG. 10B. This motion creates a wider “thick line” scan pattern, forming a scanning area 1050 that covers the full treatment area 790, as shown in FIG. 10A.

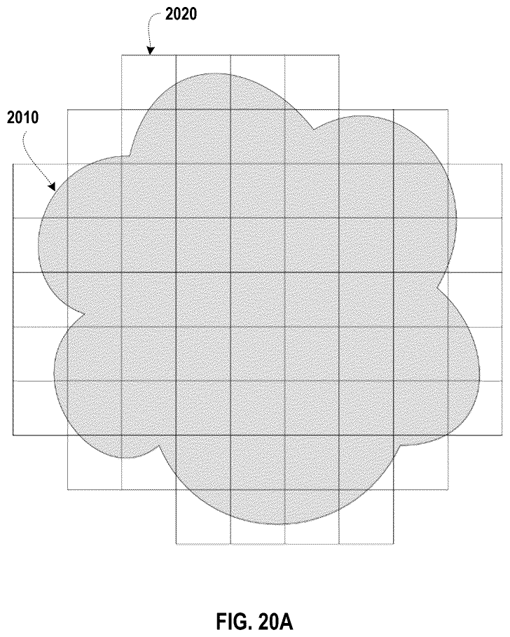

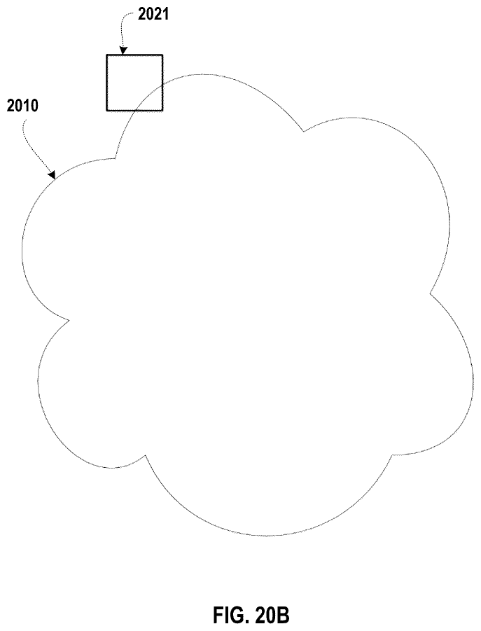

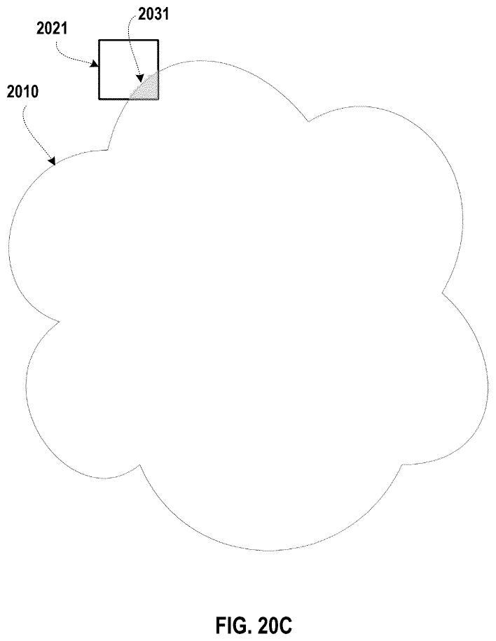

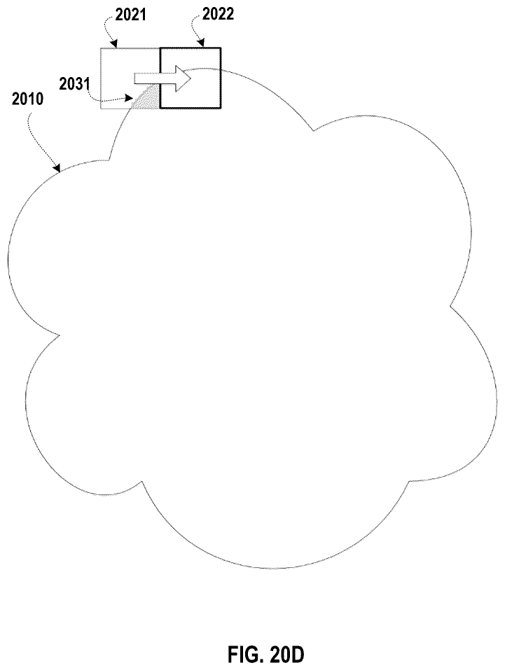

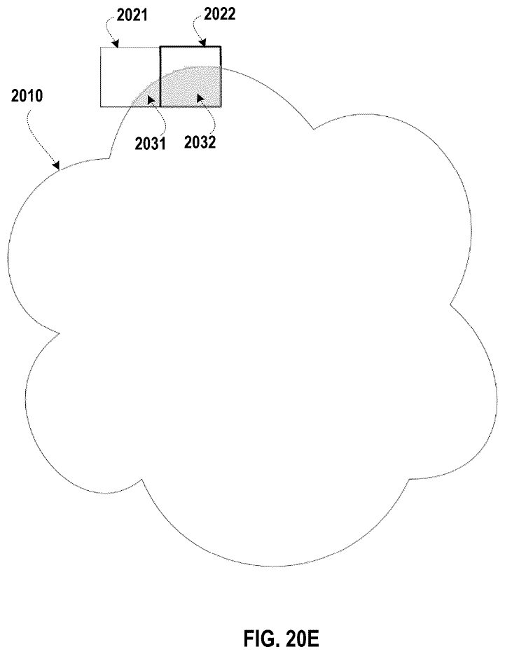

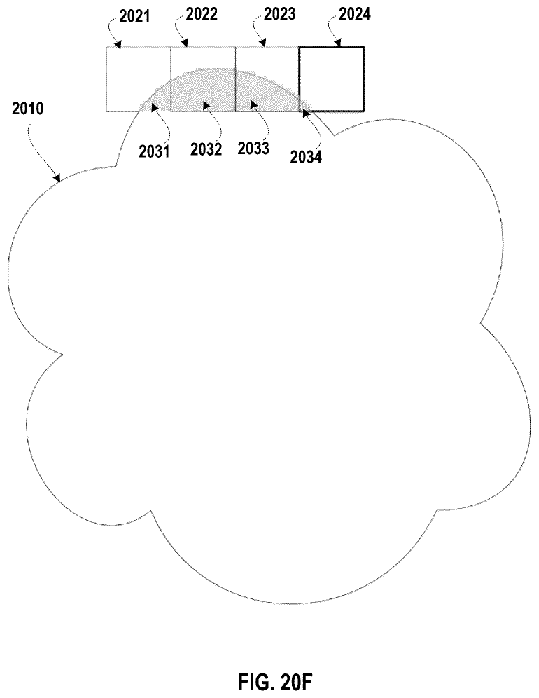

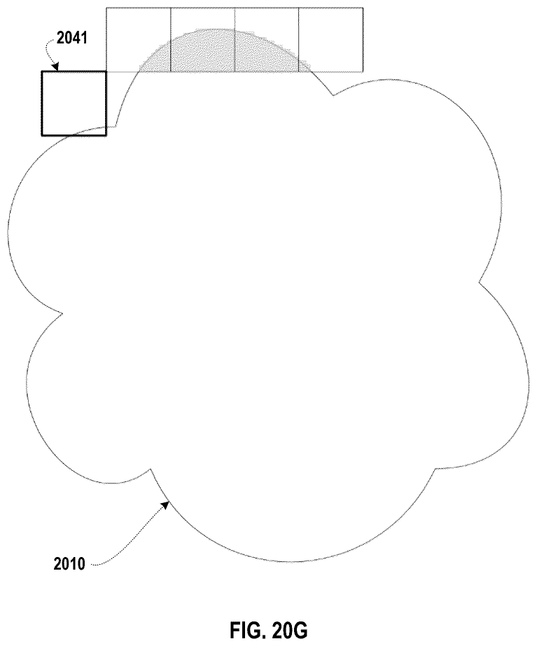

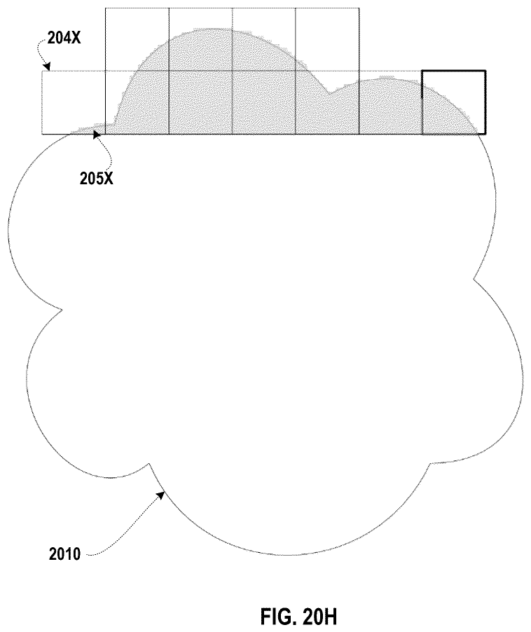

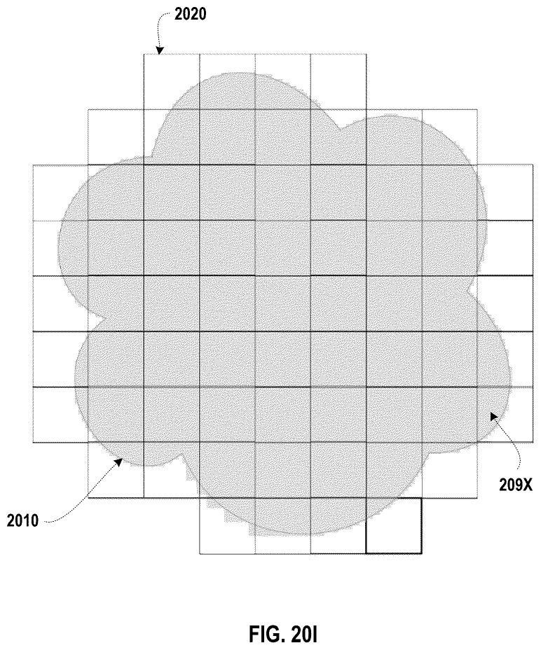

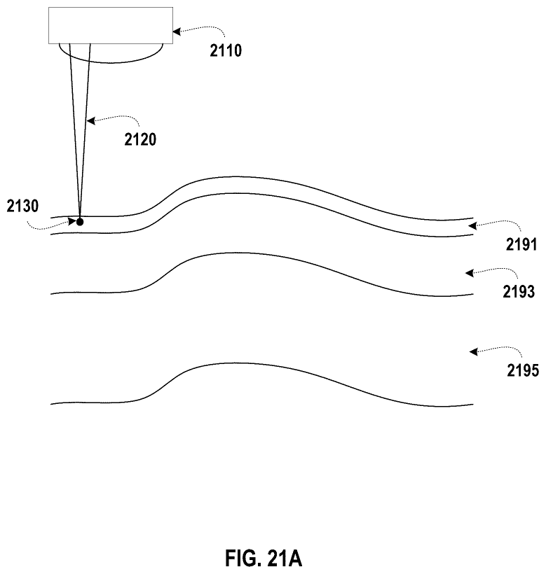

In some other embodiments, when the imaging module 110 determines that the treatment area 158 is relatively large with respect to a galvanometer scanning area of the scanning module 158, the controller 108 can divide the treatment area 158 into multiple galvanometer scanning areas and control the scanning module 158 to scan the multiple galvanometer scanning areas one by one. FIGS. 20A-20I provide examples of scanning multiple galvanometer scanning areas of a large treatment area by the medical treatment system, according to certain embodiments of the present disclosure.

As shown in FIG. 20A, the treatment area 2010 in the X-Y plane is divided into a plurality of galvanometer scanning areas 2020 each having a rectangular or square shape. The plurality of galvanometer scanning areas 2020 can completely cover the treatment area 2010. The scanning process begins with the controller 108 controlling the scanning module 158 to position itself at the first galvanometer scanning area 2021, as shown in FIG. 20B. The galvanometer is then employed to adjust the focused laser spot within first galvanometer scanning area 2021 to perform the scanning operation with high precision to cover the first treatment portion 2031 within the first galvanometer scanning area 2021, as shown in FIG. 20C. Once the first treatment portion 2031 within the first galvanometer scanning area 2021 has been completely scanned, the controller 108 instructs the X-motor to move the scanning module 158 to the second galvanometer scanning area 2022 along the same horizontal line in the X-Y plane, as shown in FIG. 20D. The galvanometer is then employed to adjust the focused laser spot within second galvanometer scanning area 2022 to perform the scanning operation with high precision to cover the second treatment portion 2032 within the second galvanometer scanning area 2022, as shown in FIG. 20E.

This process is repeated for each subsequent galvanometer scanning areas 2023, 2024, along the first line, to scan the subsequent treatment portions 2033, 2034, as illustrated in FIG. 20F. After completing the scan of all galvanometer scanning areas 2021-2024 in the first horizontal line, the Y-motor is engaged to reposition the scanning module 158 to the starting point of galvanometer scanning area 2041 in the next horizontal line, as shown in FIG. 20G. Once the scanning module 158 is positioned at each galvanometer scanning area 204X of the second line, the process resumes, with the galvanometer scanning each treatment portion 205X within the second line sequentially, as shown in FIG. 20H.

The X-motor is used to move the scanning module 158 to successive galvanometer scanning areas 2020, while the Y-motor repositions the scanning module 158 at the end of each line to transition to the next. This pattern continues until the scanning module 158 has systematically scanned all treatment portions 209X in each line of the treatment area 2010, as illustrated in FIG. 20I. This scanning approach ensures complete and efficient coverage of the entire treatment area 2010, with the precise laser spot adjustments within each galvanometer scanning area 2020 managed by the galvanometer and the coordinated movement across lines facilitated by the X- and Y-motors. The overlapping arrangement of galvanometer scanning areas 2020 provides uniform treatment without gaps or inconsistencies across the entire treatment area 2010.

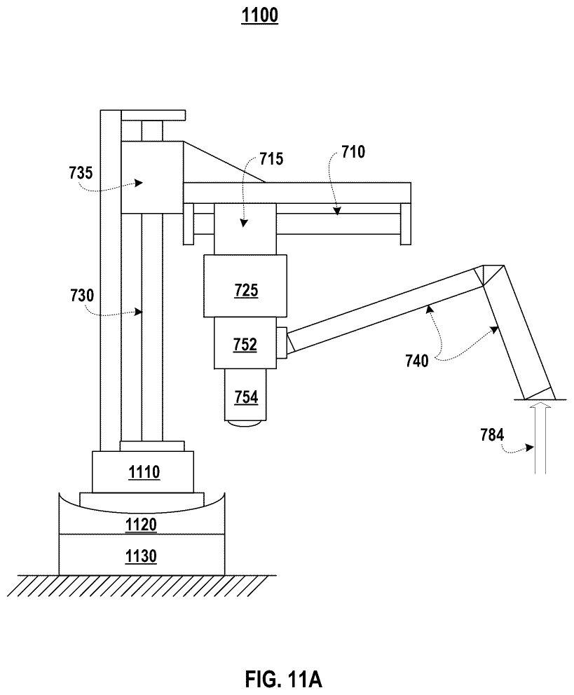

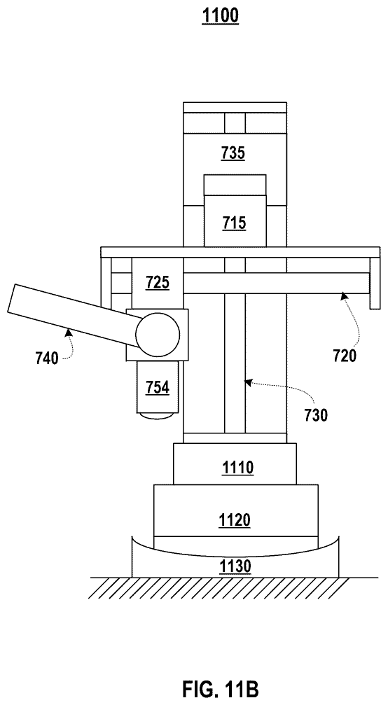

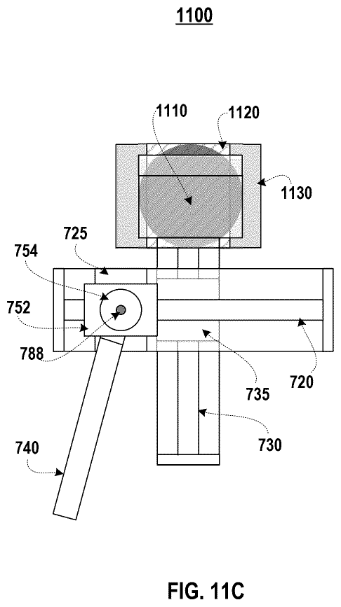

In certain embodiments, beyond the three linear degrees of freedom available in the movement of scanning module 116, additional three angular degrees of freedom can be integrated to further enhance the flexibility and precision of the system. In this configuration, the movement platform becomes a 6D movement platform. FIGS. 11A-11C present an exemplary treatment apparatus, according to some embodiments. FIGS. 11A, 11B, and 11C depict the treatment apparatus 1100 from different perspectives: FIG. 11A shows the apparatus in the X-Z plane, FIG. 11B in the Y-Z plane, and FIG. 11C in the X-Y plane, as outlined in certain embodiments of the present disclosure. In certain embodiments, as shown in FIGS. 11A-11C, the scanning control module 114 may also include an axis roll component 1110, an axis yaw component 1120, and an axis pitch component 1130. In some embodiments, the end of the third linear axis 730 (Z-axis 730) may be attached to the axis roll component 1110.

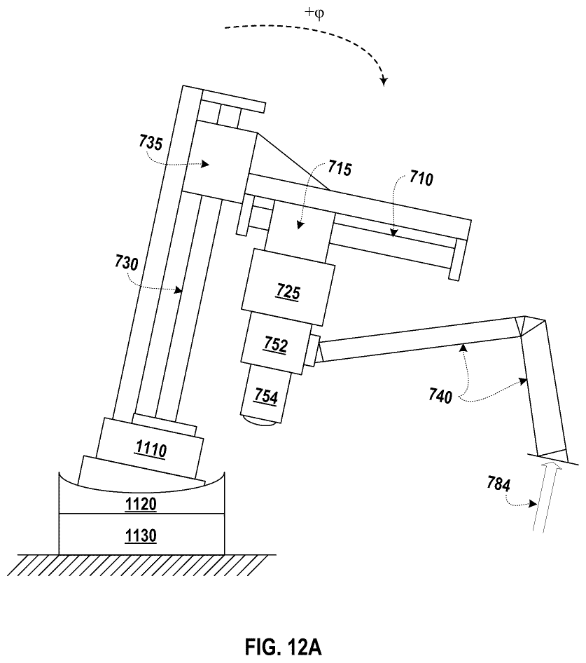

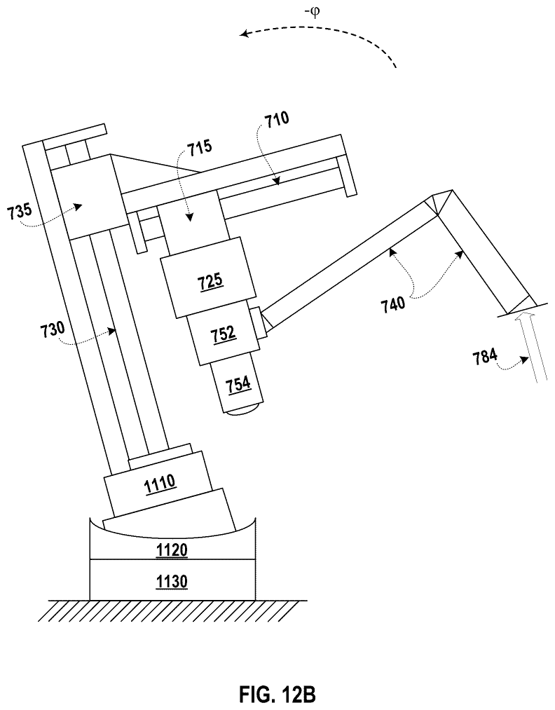

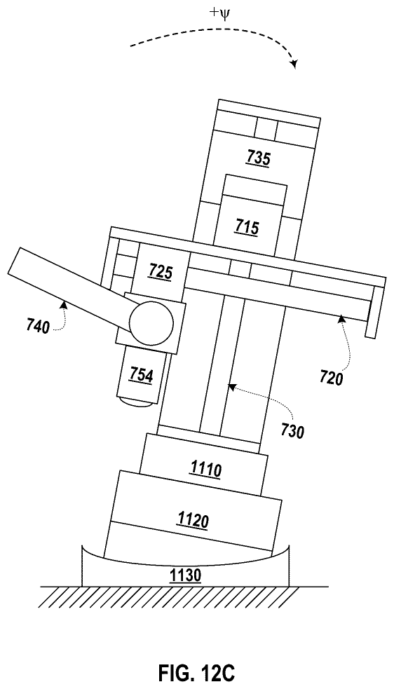

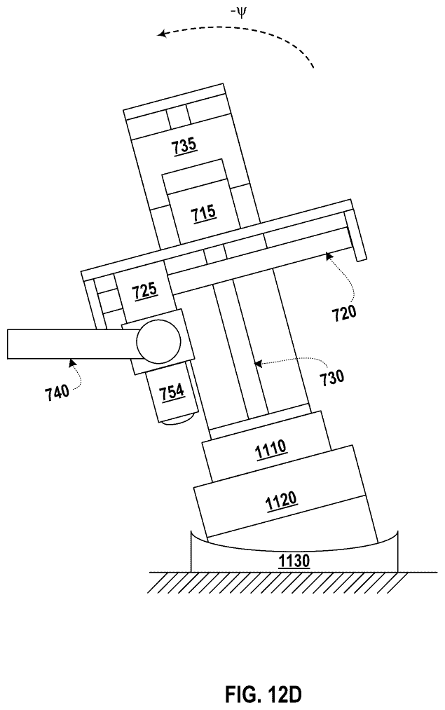

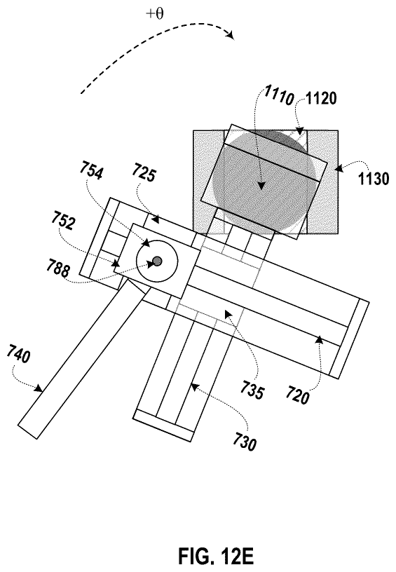

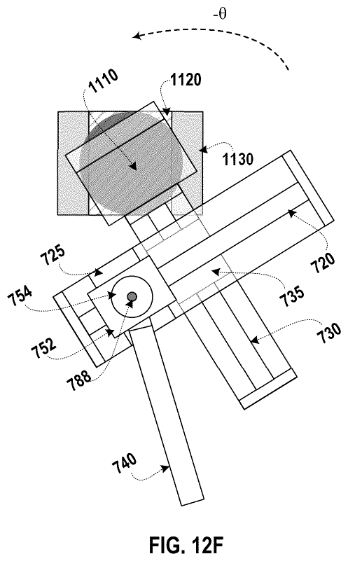

The axis roll component 1110 enables rotation around an axis parallel to the second linear axis 720 (Y-axis 720), allowing the scanning module 116 to tilt sideways, improving alignment with the target region of treatment area 790. As illustrated in FIG. 12A, the axis roll component 1110 can rotate in the positive angular direction (+φ), causing the entire 3D linear movement platform, including the first, second, and third linear axes 710, 720, 730, to rotate clockwise in the X-Z plane. In FIG. 12B, the axis roll component 1110 can rotate in the negative angular direction (−φ), which results in the entire 3D linear movement platform, including the first, second, and third linear axes 710, 720, 730, rotating counterclockwise in the X-Z plane.

In some embodiments, the yaw axis component 1120 enables rotation of an axis parallel to the first linear axis 710 (X-axis), allowing scanning module 116 to adjust its left-to-right positioning. This adjustment is crucial for reaching irregular or hard-to-access treatment areas. As illustrated in FIG. 12C, the yaw axis component 1120 can rotate in a positive angular direction (+ψ), causing the 3D movement platform—comprising the first, second, and third linear axes 710, 720, and 730—to rotate clockwise within the Y-Z plane. Conversely, as shown in FIG. 12D, the yaw axis component 1120 can rotate in the negative angular direction (−ψ), resulting in the 3D movement platform rotating counterclockwise within the X-Z plane.

In other embodiments, the pitch axis component 1130 facilitates rotation around the third linear axis 730 (Z-axis), enabling scanning module 116 to pivot up and down. This pivoting capability is essential for maintaining precise alignment with the skin's surface curvature. FIG. 12E demonstrates that the pitch axis component 1130 can rotate in a positive angular direction (+θ), causing the 3D movement platform to rotate clockwise within the X-Y plane. As shown in FIG. 12F, the roll axis component 1110 can rotate in a negative angular direction (−θ), resulting in the 3D movement platform rotating counterclockwise within the X-Y plane.

Together, these three angular movement components-yaw, pitch, and roll-combined with the three linear movement axes, provide the scanning module 116 with six degrees of freedom. This configuration enhances coverage, improves access to complex contours, and ensures optimal laser focus on various treatment areas 790. This configuration not only enhances precision but also minimizes the need for frequent repositioning of the device, improving both treatment efficiency and user comfort.

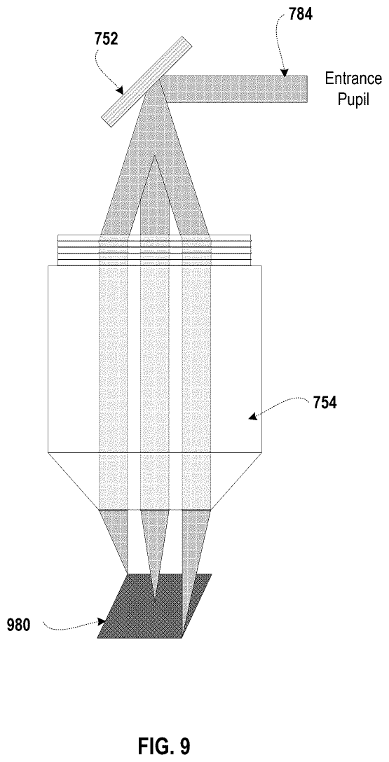

As shown in the exemplary systems depicted in FIGS. 7A-7C, 8A-8D, 9, 10A-10B, 11A-11C, and 12A-12F, the scanning module 116 is positioned on the 3D/6D movement platform, while the stage 1820 remains stationary. The system's total degrees of freedom can be up to eight, combining six degrees of freedom for the scanning module 116 with two additional degrees of freedom for the scanning galvanometer 752. In alternative embodiments not illustrated in the figures, the stage 1820 could also be mounted on the 3D/6D movement platform. For instance, in some embodiments, the scanning module 116 may remain fixed in place, while the stage 1820 is mounted on a 6D movement platform. In this case, the system would still have a maximum of eight degrees of freedom: six for the stage's movement and two for the scanning galvanometer 752.

Alternatively, both the scanning module 116 and the stage 1820 could be placed on separate 6D movement platforms. This setup would allow the stage 1820 to move over a wide range of 3D linear and/or rotational directions, albeit with lower precision. Once the stage 1820 reaches an approximate treatment position, it can pause, allowing the scanning module 116 to fine-tune the laser's positioning for precise, targeted treatment. This approach offers the advantage of coarse positioning via the stage 1820, followed by high-precision adjustments by the 3D/6D movement platform, ensuring accurate laser application on delicate treatment areas without requiring the patient to maintain a perfectly stable position throughout. In such embodiments, the system can offer a maximum of fourteen degrees of freedom. This includes six degrees of freedom for the movement of scanning module 116, six for the stage's movement, and two for the scanning galvanometer 752.

Alternatively, depending on the specific requirements of the treatment apparatus, one or more degrees of freedom for the scanning module 116 and/or the stage 1820 can be omitted. Thus, the treatment apparatus can be configured with any combination of degrees of freedom for the scanning module 116, the stage 1820, and one or two degrees of freedom for the scanning galvanometer 752.

In some embodiments, instead of utilizing three linear movement components based on a Cartesian coordinate system to achieve 3D linear motion, the 3D/6D movement platform can incorporate other movement components based on different 3D coordinate systems, such as cylindrical or spherical coordinates.

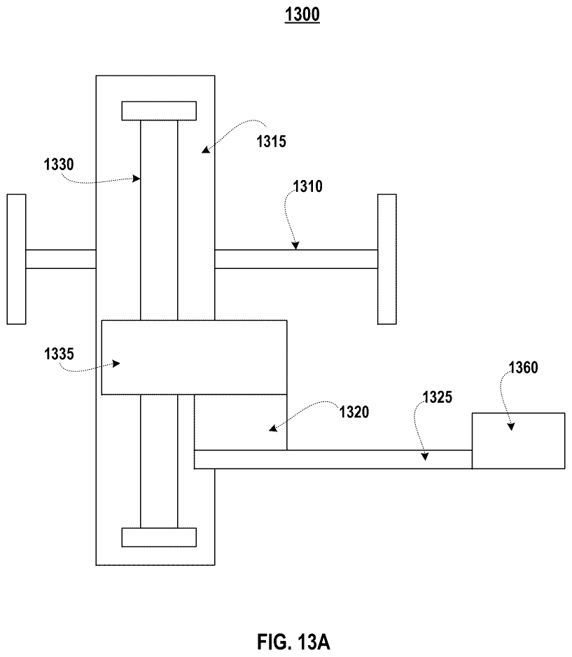

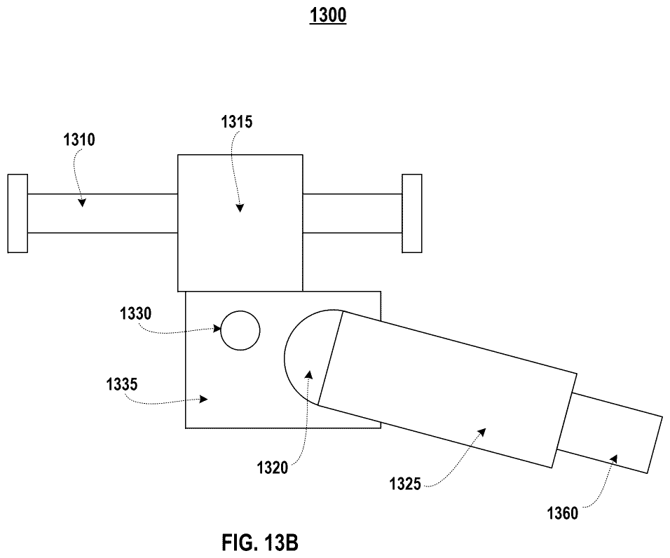

FIGS. 13A and 13B illustrate an exemplary treatment apparatus according to some embodiments of the present disclosure. FIG. 13A shows treatment apparatus 1300 in the X-Z plane, while FIG. 13B shows it in the X-Y plane. In some embodiments, treatment apparatus 1300 may feature a 3D movement platform based on a cylindrical coordinate system.

As shown in FIGS. 13A and 13B, the platform consists of two linear motion portions along two linear axes: a first linear motion portion 1315 (X-portion 1315) along the first linear axis 1310 (X-axis), and a third linear motion portion 1335 (Z-portion 1335) along the third linear axis 1330 (Z-axis). Additionally, the platform includes a rotational motion portion 1325 (θ-portion 1325) that operates along a rotation axis 1320 (θ-axis). The third linear axis 1330 is mounted on the first linear motion portion 1315, the rotation axis 1320 is mounted on the third linear motion portion 1335, and the scanning module 1360 is mounted on the rotational motion portion 1325.

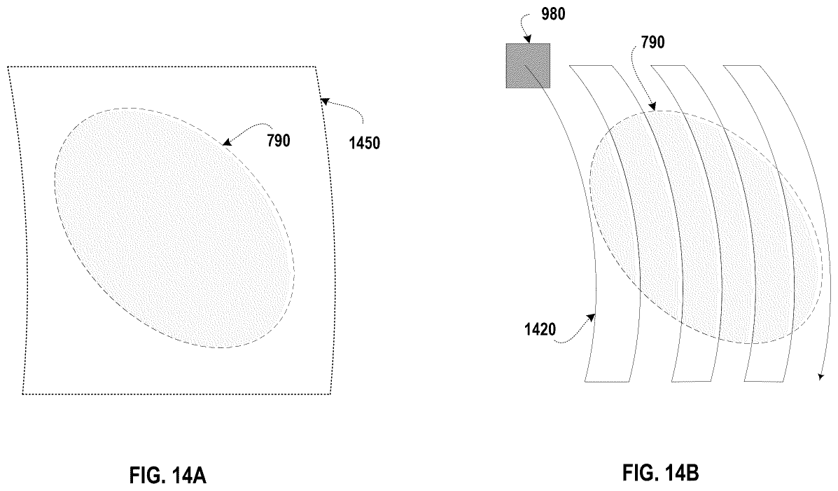

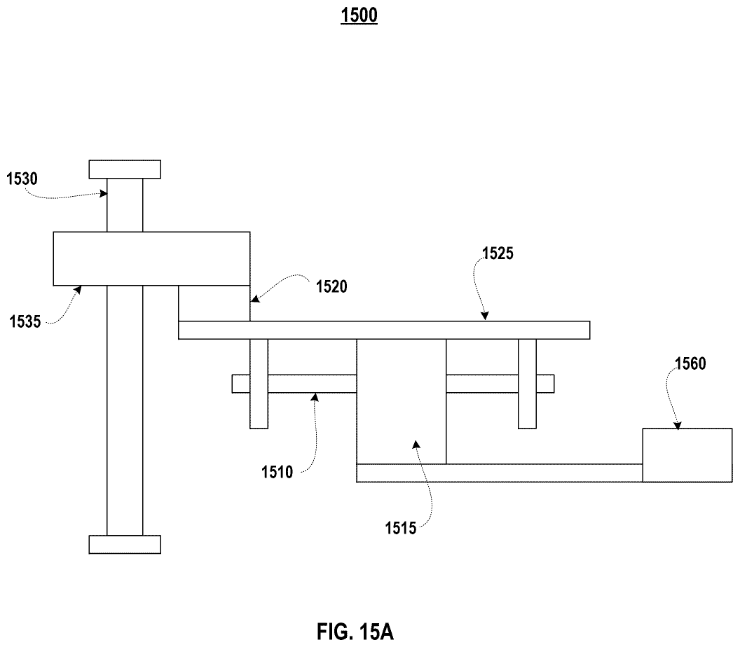

Each of the linear motion portions (1315, 1335) and the rotational portion 1325 is independently driven by a separate linear/rotary motor module, enabling movement along their respective axes—X-axis 1310, Z-axis 1330, and θ-axis 1320. In some embodiments, within the X-θ plane, the combined motion of the first linear motion portion 1315 (X-portion) along the X-direction and the rotation portion 1325 (θ-portion) along the θ-direction allows the projection area 980 of the laser beam from scanning module 1360 to move along trajectory 1420, as shown in FIG. 14B. This movement enables the broader “thick line” scanning pattern, creating a scanning area 1450 that covers the entire treatment area 790 in the X-θ plane, as illustrated in FIG. 14A.

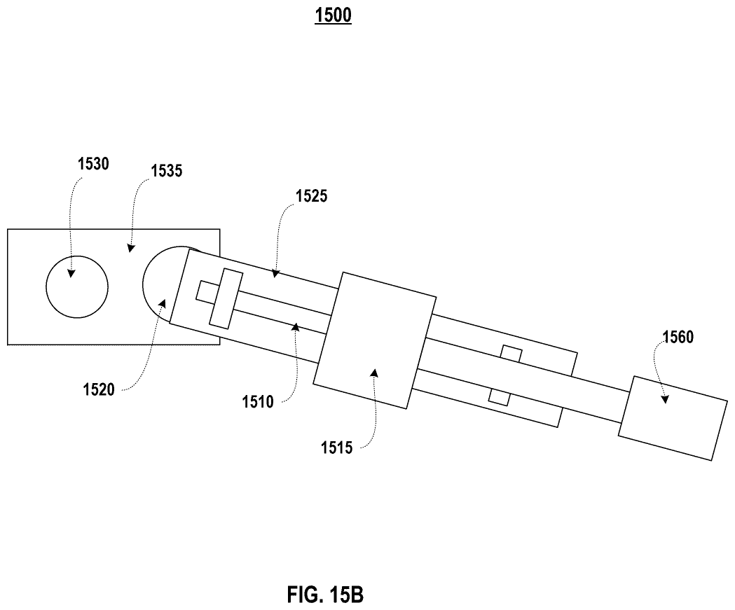

FIGS. 15A and 15B depict another exemplary treatment apparatus, according to some embodiments of the present disclosure. FIG. 15A shows treatment apparatus 1500 in the X-Z plane, while FIG. 15B shows it in the X-Y plane. In some embodiments, treatment apparatus 1500 includes a 3D movement platform based on a second cylindrical coordinate system.

As shown in FIGS. 15A and 15B, this 3D movement platform includes two linear motion portions along two axes: a first linear motion portion 1515 (R-portion) along the radial linear axis 1510 (R-axis), and a third linear motion portion 1535 (Z-portion) along the third linear axis 1530 (Z-axis). The platform also includes a rotational motion portion 1525 (θ-portion) along the rotation axis 1520 (θ-axis). The rotation axis 1520 is mounted on the third linear motion portion 1535, the first linear motion portion 1515 is mounted on the rotation portion 1525, and the scanning module 1560 is mounted on the first linear motion portion 1515.

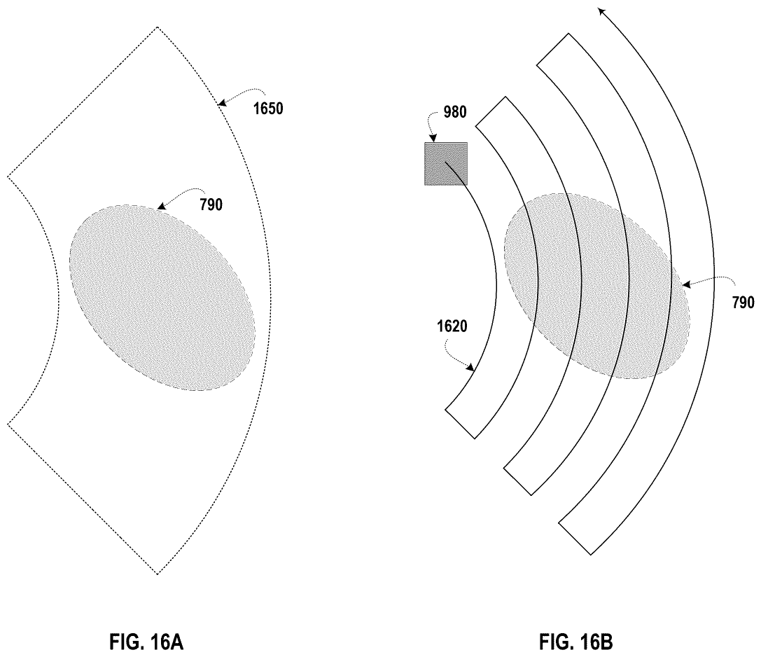

Each of the linear motion portions (1515, 1535) and the rotation portion 1525 is independently driven by separate linear/rotary motor modules, enabling independent movement along the R-axis, Z-axis, and θ-axis, respectively. In some embodiments, within the R-θ plane, the combined motion of the radial linear motion portion 1515 (R-portion) along the radial direction (R-direction) and the rotation portion 1525 (θ-portion) along the rotational direction (θ-direction) allows the projection area 980 of the laser beam from scanning module 1560 to follow trajectory 1620, as shown in FIG. 16B. This results in a broader “thick line” scanning pattern that forms a scanning area 1650, covering the entire treatment area 790 in the R-θ plane, as shown in FIG. 16A.

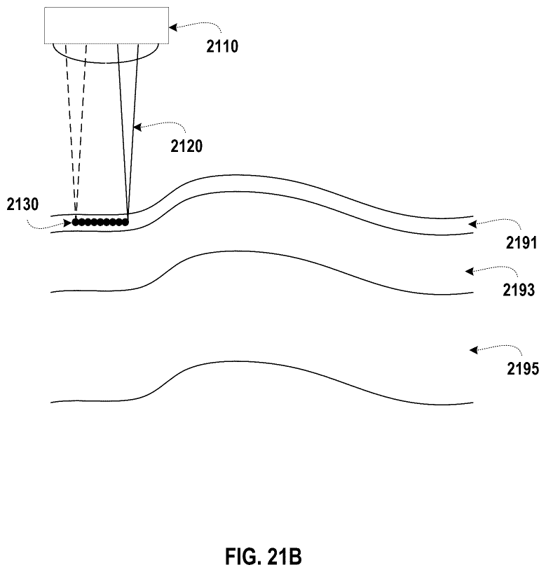

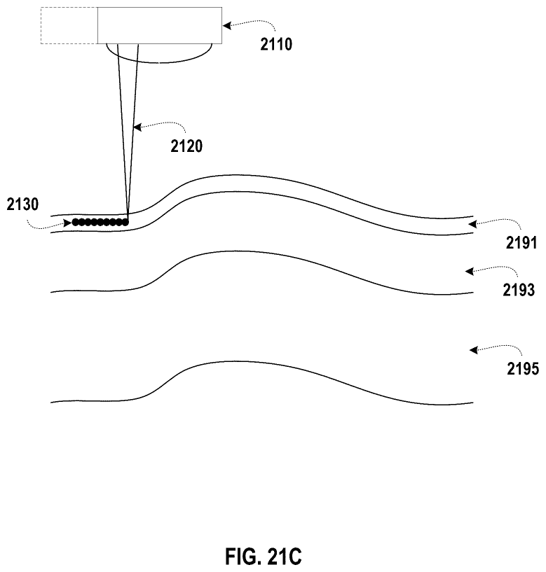

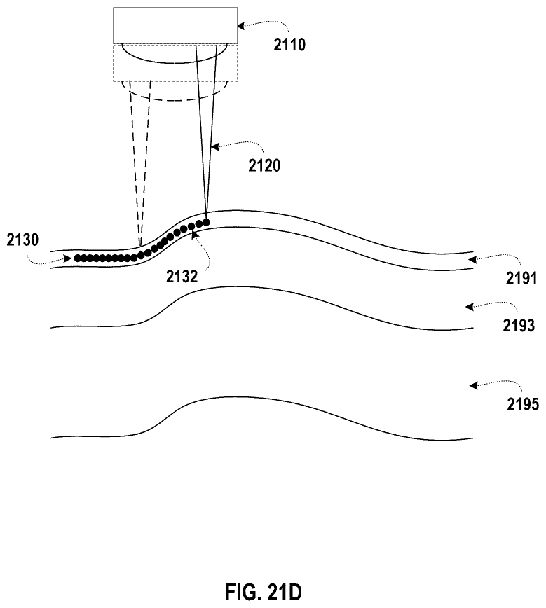

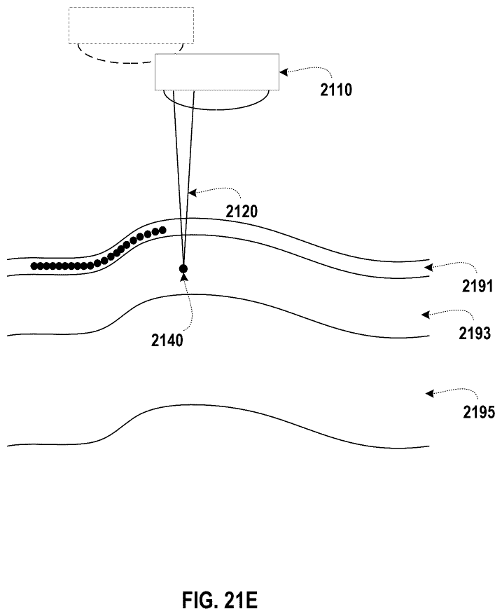

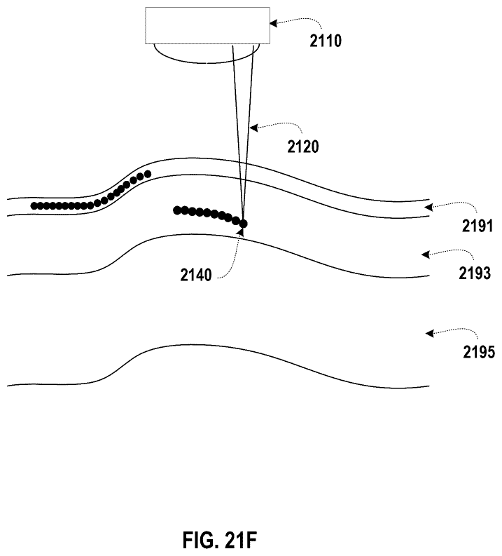

Referring to FIGS. 21A-21F, examples of a laser forcing method for treating different areas at different depths of skin using the medical treatment system are illustrated, according to certain embodiments of the present disclosure. As shown in FIG. 21A, the treatment area of the skin of a patient can include epidermis 2191, dermis 2193, and subcutaneous structure 2195 at different depths from the surface of the skin of the patient.

The scanning process begins with the controller 108 controlling the scanning module 158 to position the laser head 2110 including the lens at an initial position. The galvanometer is then employed focus laser beam 2120 to from a first focused laser spot 2130 at a first depth within epidermis 2191, as shown in FIG. 21A. The galvanometer can then scan along the planar surface to adjust the focused laser beam 2120 to form a series of first focused laser spot 2130 at the first depth within epidermis 2191, as shown in FIG. 21B. When the galvanometer completes its galvanometer scanning area, the controller 108 instructs the X/Y-motor to move the scanning module 158 to the next position in the X-Y plane, and the galvanometer repeats the scanning process, as shown in FIG. 21C. When the distance measuring module 112 detects the skin surface is not even in a horizontal plane but include bumps and depressions, the controller 108 instructs the Z-motor to move the scanning module 158 along the vertical direction during the scanning process of the galvanometer to adjust the locations of the formed second focused laser spots 2132, such that the series of second focused laser spots 2132 have different second depths in the vertical direction, as shown in FIG. 21D.

Referring to FIG. 21E, when the controller determines that the focus laser spot 2140 need to penetrate the epidermis 2191 to reach dermis 2193, the controller 108 can also instruct the X/Y/Z-motor to move the scanning module 158 to change location of the laser head 2110 in the 3D space, such that the formed third focused laser spot 2140 at a third depth within the dermis 2193. Similarly, the X/Y/Z-motor can move the scanning module 158 while the galvanometer can scan to adjust the focused laser beam 2120 to form a series of third focused laser spot 2140 sequentially at different third depths within dermis 2193, as shown in FIG. 21F.

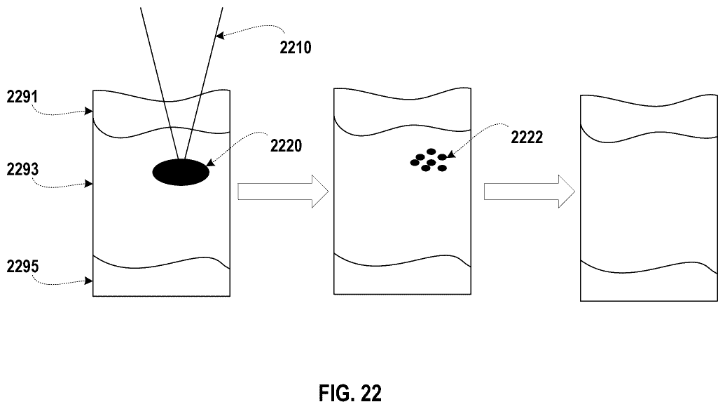

FIG. 22 depicts an example of a laser-based method for treating dermal pigmentation, according to certain embodiments of the present disclosure. As shown, the laser-based method for treating dermal pigmentation involves precisely targeting the pigment 2220 located in the dermis 2293, below the epidermis 2291 and above the subcutaneous structure 2295. First, a specialized laser 2210 is calibrated to emit a specific wavelength that selectively focuses on the pigment 2220 without damaging surrounding tissues. The pigment 2220 absorbs the laser energy, which causes it to break into smaller fragments 2222 through a process known as photothermolysis. Once fragmented, these pigment fragments 2222 are naturally processed and absorbed by the skin's immune system, primarily through macrophage activity. Over time, this absorption results in a gradual fading of the pigmentation, leading to a more even skin tone.

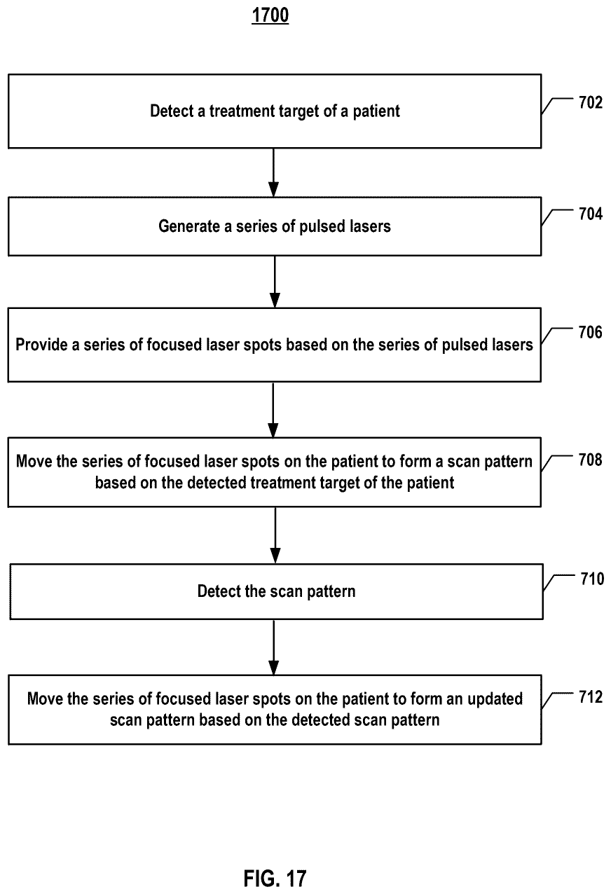

FIG. 17 is a flowchart illustrating method 1700 for performing medical treatment utilizing focused laser spots, according to some embodiments. It should be noted that the operations in method 1700 are not exhaustive; additional operations may be performed before, after, or between the illustrated steps. Moreover, some operations can be executed concurrently or in a different sequence than shown in FIG. 17.

The method begins at operation 1702, where the treatment area of the patient is detected. In some embodiments, detection data indicating the treatment area is collected. This operation is carried out by imaging module 110 of system 100 prior to the medical treatment. In some embodiments, imaging module 110 can be a camera designed to capture precise samples of the skin and accurately identify the treatment area. In certain embodiments, the camera may be a hyperspectral imaging camera that captures images of the treatment target across multiple spectral bands, providing diagnostic insights that aid in treatment planning. Detection of the treatment area may also be carried out manually by a human operator in some cases.

In other embodiments, the detection data may be processed by a trained AI model, which automatically identifies and classifies the type of lesion or condition to be treated. This enables a targeted and informed treatment approach. The detection data can be processed to enable multiple layers of feature recognition to support more accurate treatment. For example: A texture feature recognition model can be used to analyze surface texture patterns, helping identify defects or irregularities in the skin. This is particularly useful for distinguishing treatment areas based on skin texture characteristics. A shape feature recognition model can identify and verify specific shape features of the treatment area, including confirming surface defects. A scan pattern recognition model can detect and confirm scan patterns in real-time, allowing for adaptive scanning based on the detected patterns. A color feature recognition model can analyze and confirm color variations, which is critical for differentiating pigmentation and other color-based characteristics of the treatment area, ensuring precise targeting of the laser. These detection data processing models work together to enhance the system's ability to adjust treatment parameters in real time, ensuring high precision and effectiveness when targeting specific skin conditions.

Method 1700 then progresses to operation 1704, as shown in FIG. 17, where a set of pulsed lasers are generated. In some embodiments, each pulsed laser may consist of a set of bursts. This operation is carried out by laser source 102 of system 100, which is capable of providing a flexible range of pulse lengths and repetition frequencies to suit various treatment requirements. For instance, the laser pulse duration may range from 1 ns to 1,000 ns, suitable for treatments requiring relatively longer pulses. Alternatively, the pulse duration can be set between 1 ps to 1,000 ps, offering greater precision for delicate procedures. For ultra-precise applications, pulse durations can be adjusted to 50 fs to 1,000 fs, minimizing thermal impact on surrounding tissues and ensuring minimal damage to healthy skin. In some embodiments, the repetition frequency of the pulsed lasers is adjustable, ranging from 1 Hz to 50 MHz, providing flexibility in pulse delivery speed to match different treatment protocols and optimize therapeutic outcomes.

Based on the treatment area detected in operation 1702, the pulsed lasers are activated only in designated regions of the treatment area, preventing unnecessary exposure to non-targeted regions and enhancing patient safety. In some embodiments, the pulsed lasers can be modulated dynamically, activating only at specific points as needed. This approach, similar to the precision of embroidery, enables selective treatment of different target spots within the treatment area. The system can determine whether each specific location requires laser exposure, ensuring a targeted treatment approach. Moreover, the intensity, frequency, duration, and/or repetition frequency of the pulsed lasers can be adjusted in real time, based on the detection data, to match the specific needs of the treatment area. This dynamic adjustment ensures high precision and optimizes therapeutic effectiveness.

Finally, method 1700 proceeds to operation 1706, as shown in FIG. 17, where a set of focused laser spots are delivered, based on the pulsed lasers. In some embodiments, to create the series of focused laser spots, each pulsed laser is focused such that the dimension of each focused laser spot falls within the range of 0.1 μm to 500 μm. Operation 1706 is implemented by the scanning control module 114 and distance measuring module 112 of system 100.

In certain embodiments, the distance measuring module 112 may incorporate either a range sensor or Light Detection and Ranging (LiDAR) technology. These sensors are capable of accurately determining the distance between scanning module 116 and the treatment area, ensuring the laser can be precisely focused and aligned for optimal treatment effectiveness. The range sensor is well-suited for measuring short distances, offering quick and direct readings, while LiDAR excels in providing greater accuracy, especially when capturing three-dimensional spatial data over larger or more intricate surfaces.

Alternatively, some embodiments may equip the distance measuring module 112 with a spectral confocal displacement sensor. This type of sensor facilitates highly accurate distance measurements, even in challenging conditions such as when the treatment area has significant tilt or curvature. The spectral confocal displacement sensor is particularly effective on reflective, transparent, or semi-transparent surfaces, providing impressive tolerance for angular variations. It allows for precise measurements without the need for strict alignment adjustments, and can maintain accuracy even if there are small vibrations or slight shifts in the treatment area during the measurement process.

In certain embodiments, the scanning control module 114 can utilize data from the distance measuring module 112 to accurately assess the height variations across different points in the treatment area. This data is then used to dynamically adjust the position of the laser focusing head of measuring module 112, allowing for real-time modifications. Such continuous adjustments enable precise control over the focal depth of the laser on the skin, ensuring that the treatment is focused on the desired skin layer for maximum therapeutic effectiveness.

As method 1700 progresses to operation 1708, as shown in FIG. 17, the next step involves shifting the series of focused laser spots across the patient's skin, thus forming a scan pattern based on the detected treatment area. In some embodiments, this movement of the focused laser spots is achieved by altering the directions in which the pulsed laser is directed towards the patient. For instance, this can be done through adjustments made by scanning module 116 of scanning control module 104. In some embodiments, the positioning of the patient is adjusted during the medical procedure to move the series of focused laser spots, which can be achieved through the use of the disclosed 3D/6D movement platform as described earlier. Additionally, in some cases, a control instruction defining the scan pattern is provided by controller 108 of system 100, based on the detection data gathered by imaging module 110. In other embodiments, the positions of the focused laser spots on the patient may be calibrated beforehand, ensuring that the movement of these spots follows the pre-established calibration settings. Furthermore, in certain instances, some of the focused laser spots are positioned at varying depths beneath the patient's skin to optimize the treatment process.

As method 1700 advances to operation 1710, as depicted in FIG. 17, the next step involves detecting the scan pattern during the course of the medical treatment. This operation is carried out by imaging module 110 during the procedure. In some embodiments, the scan pattern detection may be performed manually by a human operator.

Moving on to operation 1712, as illustrated in FIG. 17, the series of focused laser spots is repositioned on the patient to form an updated scan pattern based on the detected scan pattern. In some embodiments, updated detection data is provided to controller 108 by imaging module 110, based on the scan pattern's detection. In certain cases, the size and/or shape of the scan pattern may be modified to generate the updated scan pattern. Alternatively, adjustments to the path of the movement of the focused laser spots across the patient can be made, resulting in the formation of the updated scan pattern. Additionally, during the medical treatment, the power applied by the focused laser spots to the patient may be modified. For instance, power adjustments can be achieved by altering the laser source's power or changing the number of bursts within each pulsed laser. In another example, adjustments can be made by modifying the speed and/or repetition rate of the focused laser spots' movement.

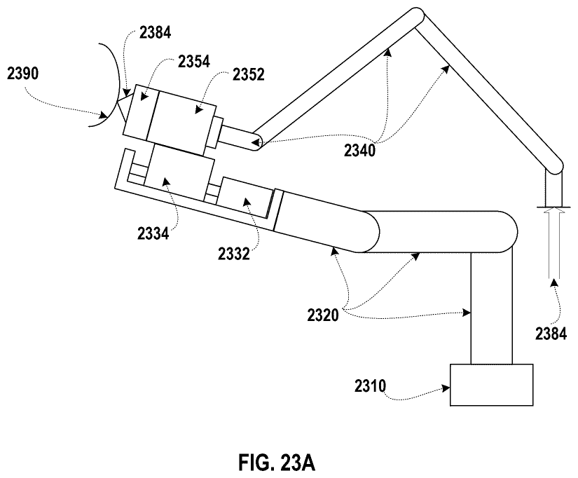

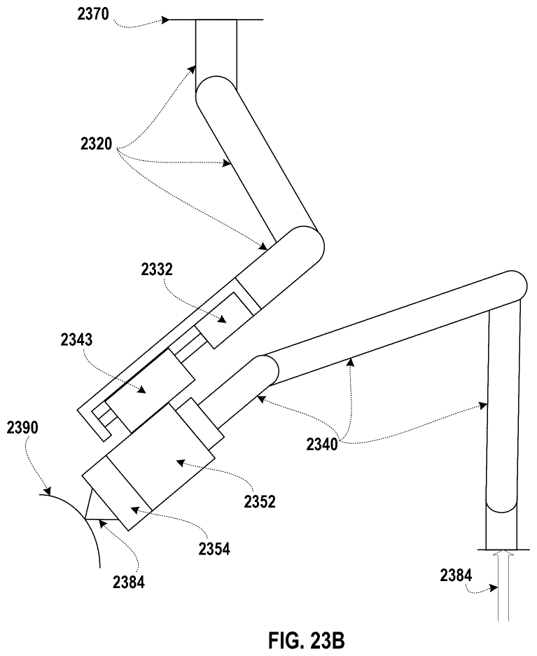

In certain embodiments, the 6D movement platform can include a robot arm. FIGS. 23A and 23B present exemplary treatment apparatus, according to some embodiments. In certain embodiments, as shown in FIGS. 23A and 23B, the treatment apparatus may include a light guide 2340, which is configured to transmit laser beam 2384 from a laser source to a scanning module containing a scanning galvanometer 2352 and a laser objective lens 2354. In certain embodiments, the light guide 2340 can include multiple sections each having the ability to rotate along one or more axes or, such that the scanning module can efficiently and dynamically adjust the beam path before passing it through the field lens 2354 to the treatment area 2390. The length of each section of the light guide 2340 can be tailored to meet specific treatment requirements, with the ability to adjust within a range that suits the conditions of the procedure.

As shown in FIGS. 23A and 23B, the treatment apparatus may further include a robotic arm 2320, such as a 6-axis robotic arm. Similarly, robotic arm 2320 can include multiple sections each having the ability to rotate along one or more axes. The length of each section of the robotic arm 2320 can be tailored to meet specific treatment requirements, with the ability to adjust within a range that suits the conditions of the procedure. In some embodiments as shown in FIG. 23A, robotic arm 2320 can be mounted on a station 2310, such as a movable station or a fixed station. In some embodiments as shown in FIG. 23B, robotic arm 2320 can be mounted on a suspended configuration 2370, such as a movable plate along a track on a ceiling or a wall, significantly expanding its range of motion and workspace flexibility.

In certain embodiments, a motor 2332 and a slider 2334 can be amounted on an end of the robotic arm 2320, and scanning galvanometer 2352 can be mounted on the slider 2334, as shown in FIGS. 23A and 23B. In certain embodiments, motor 2332 can be a high-precision linear motor, allowing for precise movement of slider 2332 along the optical axis. The motor 2334, the slider 2332, the scanning galvanometer 2352, and the field lens 2352 can work together to direct and focus the laser beam 2384 transmitted by light guide 2340.

The robotic arm 2320 can smoothly and dynamically move and orient the scanning module to allow the focused laser spot 2388 to be positioned on treatment area 2390. For example, the 6-axis robotic arm 2320 enables field lens 2354 along along complex curved treatment surface, continuously adjusting the orientation of focused laser spot 2388 to maintain perpendicular alignment with the local tangent plane at every treatment point of treatment area 2390. This ensures uniform energy delivery, enhancing precision and optimizing therapeutic effectiveness, especially on irregular or contoured treatment area 2390, minimizing collateral damage to surrounding tissue.

Additionally, the linear motor 2332 can provide real-time focal depth control, allowing precise adjustments to the laser's penetration depth. This feature is crucial for targeting different tissue layers with high accuracy, enhancing both treatment efficacy and safety. The combined motion control of the robotic arm 2320 and linear motor 2332 ensures superior precision, flexibility, and adaptability, making it ideal for advanced medical laser treatments.

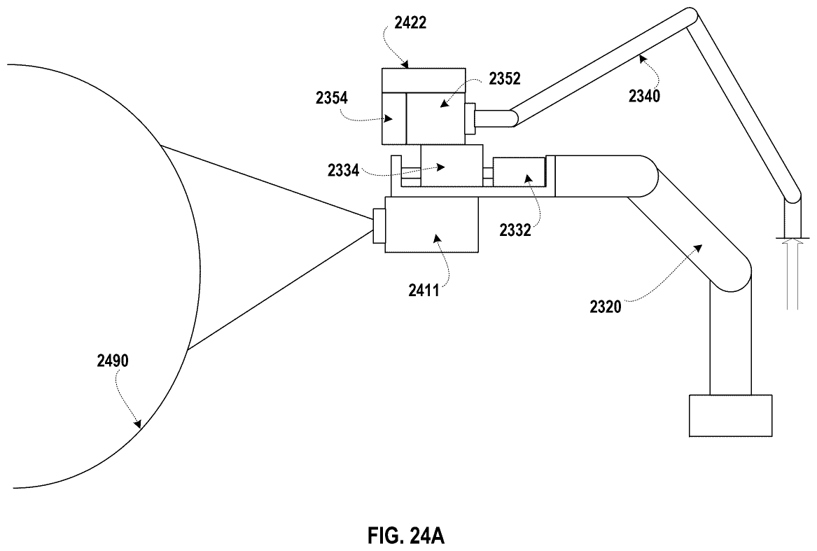

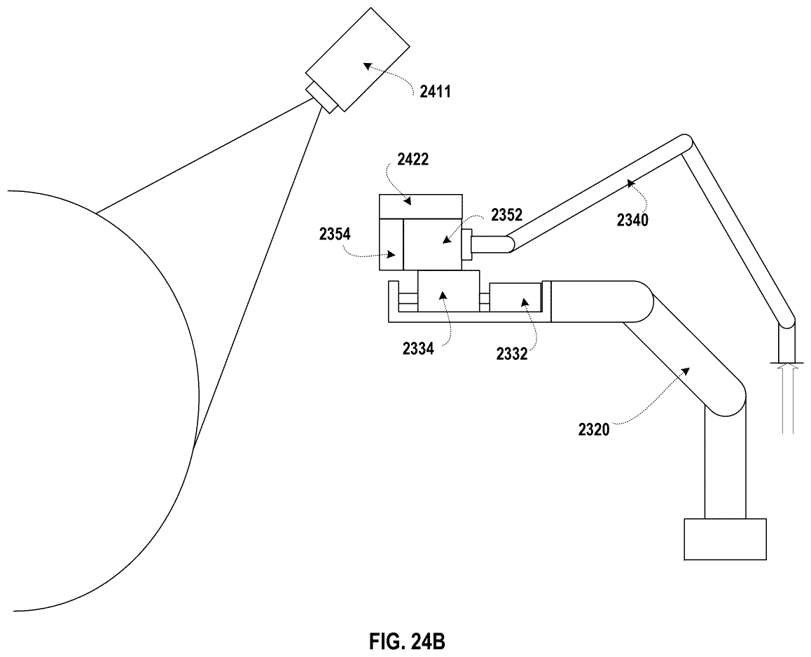

In certain embodiments as shown in FIG. 24A, a displacement sensor 2422 can be positioned on the scanning galvanometer 2352 and/or the field lens 2354. In some implementations, displacement sensor 2422 can be a spectral confocal displacement sensor. Using the dispersion-based confocal principle, the displacement sensor 2422 enables high-precision distance measurements from the field lens 2354 and the surface point of treatment area 2490 on the optical axis of the field lens 2354, without being affected by optical aberrations of any optical component in the optical system. The displacement sensor 2422 can maintain accuracy even when the surface of the treatment area 2490 is tilted at steep angles. The displacement sensor 2422 can perform exceptionally well on reflective, transparent, or semi-transparent surfaces and offers a strong tolerance to angular variations, such that high-precision distance measurements can be achieved without the need for strict angle adjustments, and the displacement sensor 2422 can be less sensitive to vibrations or slight movements of the treatment area 2490 during measurement operations.

In certain embodiments, as shown in FIGS. 24A and 24B, a depth camera 2411 can be included in the system to enhance real-time surface mapping and treatment precision. By capturing 3D depth information of the treatment area 2490, the depth camera 2411 can allow the robotic arm 2320 to dynamically adjust its position and orientation, ensuring that the laser remains perfectly perpendicular to each local region of the treatment area 2490 at all times. In some implementations, the depth camera 2411 can enable automatic surface adaptation, optimized focal depth control, and enhanced safety and consistency. For example, the robotic arm 2320 can precisely follow complex contours without manual adjustments based on input from depth camera 2411. Further, the depth data generated by depth camera 2411 can be used to adjust the position of slider 2334, ensuring the laser maintains accurate penetration depth across varying surface geometries. In addition, by continuously analyzing surface variations using depth camera 2411, the liner motor 2332 and/or robotic arm 2320 can automatically compensate for patient movement or unexpected deviations, maintaining consistent energy delivery. Therefore, by integrating the depth camera 2411, the medical treatment system can achieve higher precision, improved efficiency, and better treatment outcomes, making it ideal for adaptive laser-based medical applications.

It is noted that, there are two common configurations for integrating the depth camera 2411 with the robotic arm 2320. In an Eye-On-Hand configuration as shown in FIG. 24A, the depth camera 2411 is attached to an end effector of robotic arm 2320, moving along with the laser system. This allows the depth camera 2411 to capture real-time 3D depth data from different angles, dynamically adjusting the motion of robotic arm 2320, as well as the laser focal depth based on the treatment surface 2490. The Eye-On-Hand configuration can have many advantages, such as flexible viewing angles, precise surface tracking, and better handling of dynamic environments. For example, the depth camera 2411 moves with the robotic arm 2332, thus providing continuous scanning from various perspectives, ensuring real-time adaptation to surface variations, improving laser alignment and penetration accuracy, and enabling auto compensation for patient movement in real-time. That is the Eye-On-Hand configuration is ideal for dynamic, real-time adjustments during treatment.

In an Eye-To-Hand configuration as shown in FIG. 24B, as shown in FIG. 24B, the depth camera 2411 can be mounted externally, such as separated and detached from robotic arm 2320. This provides a fixed, wide-field view of the treatment area 2490, enabling pre-scanning and real-time monitoring of the operations of the robotic arm 2332 and/or slider 2334. The Eye-To-Hand configuration can have many advantages, such as wider coverage, stable reference frame, and simpler hardware integration. For example, the detached depth camera 2411 can capture a larger region of treatment area 2490 at once, useful for global positioning and treatment planning. Further, separated from robotic arm 2320, the detached depth camera 2411 can provide consistent depth measurements without being affected by the movements of robotic arm 2320. Additionally, removing depth camera 2411 from robotic arm 2322 can reduce the carrying weight on the robotic arm 2322, maintaining its speed and efficiency. That is, the Eye-To-Hand configuration is suited for pre-treatment mapping and overall monitoring.

In some other implementations not shown in FIGS. 24A and 24B, more than one depth cameras can be applied. For example, a hybrid approach combining both configurations can offer the best of both of the precise local scanning with the Eye-On-Hand depth camera and the global positioning with the Eye-To-Hand Eye-On-Hand depth camera.

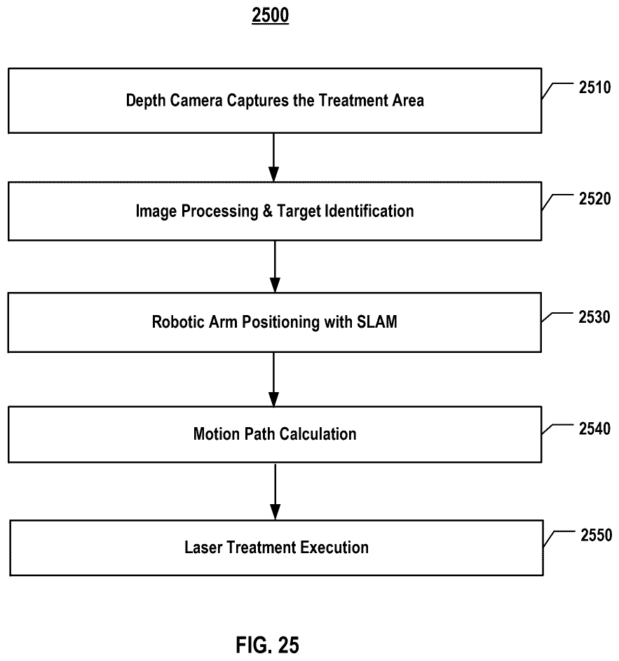

FIG. 25 is a flowchart illustrating method 2500 for performing medical treatment utilizing focused laser spots, according to some embodiments. In certain embodiments, method 2500 are applied by medical treatment apparatus shown in FIG. 24A or 24B. It should be noted that the operations in method 2500 are not exhaustive; additional operations may be performed before, after, or between the illustrated steps. Moreover, some operations can be executed concurrently or in a different sequence than shown in FIG. 25.

The method begins at operation 2510, in which a depth camera captures an image of a treatment area. In certain implementations, the depth camera can be configured as either an Eye-On-Hand depth camera mounted on the robotic arm's end effector, or an Eye-To-Hand depth camera externally fixed. The depth camera can capture a detailed 3D image of the treatment area. The depth camera collects depth and RGB data, creating a point cloud or depth map that represents the surface geometry. The generated information serves as the foundational dataset for identifying the regions requiring treatment. In certain implementations, advanced depth cameras, such as structured light or time-of-flight (ToF) sensors, ensure high accuracy even in dynamic environments, compensating for subtle patient movements.

The method processes operation 2520 of image processing and target identification. In operation 2520, the depth data is fed into an image recognition algorithm, which performs segmentation and pattern analysis to identify the boundaries of the treatment area. Techniques such as convolutional neural networks (CNNs) or traditional computer vision methods (e.g., edge detection and contour mapping) are employed to precisely locate and label the target regions. The system generates a set of 3D coordinates corresponding to the surface points where treatment is needed. To enhance accuracy, this step may involve filtering out irrelevant features, correcting for ambient lighting conditions, and compensating for minor anatomical variations.

The method processes operation 2530, in which the robotic arm is positioned using simultaneous localization and mapping (SLAM). The robotic arm can determine its exact position relative to the treatment surface during operation 2530. The SLAM algorithm processes visual and depth inputs to create a continuously updated map of the environment while tracking the movement of the robotic arm. This real-time spatial awareness ensures that the robotic arm maintains a stable and precise orientation, even if the treatment setup shifts slightly. The use of SLAM is particularly advantageous in dynamic clinical environments where fixed reference points may change.

The method processes operation 2530, in which the motion path of laser head can be calculated. Specifically, with the coordinates of the treatment area and the current position established for the robotic arm, the system can calculate an optimal motion trajectory. For example, inverse kinematics (IK) algorithms can be applied to determine joint angles that will move the laser head smoothly along the treatment surface. The trajectory of laser head is designed to keep the laser beam perpendicular to the tangent plane of the surface at all times, maximizing energy delivery efficiency and minimizing thermal damage to surrounding tissues. The system can also adjust the linear motor controlling the focal depth, ensuring the laser precisely targets the desired tissue layer, regardless of surface curvature.

The method processes operation 2530, in which Laser Treatment Execution Once the motion path is finalized, the robotic arm follows the calculated trajectory to apply the laser treatment. The galvanometer and field lens, mounted on the linear motor's slider, rapidly adjust the beam's position and focus. Throughout this process, the depth camera continues to capture real-time images, allowing for dynamic adjustments based on patient movement or unexpected variations in the treatment surface. Safety protocols, such as thermal monitoring and emergency stop mechanisms, are integrated to protect both the patient and the equipment. The continuous feedback loop ensures consistent energy delivery, optimal treatment outcomes, and a seamless user experience for the clinician.

Another facet of the disclosure pertains to a non-transitory computer-readable medium that stores instructions. When executed, these instructions prompt one or more processors to carry out the previously discussed methods. The computer-readable medium can take the form of a volatile or non-volatile storage device, such as magnetic, semiconductor, tape, optical, removable, or non-removable media, or any other suitable computer-readable storage device. For instance, the computer-readable medium could be a disc or flash drive. The computer instructions may be stored on such disc or flash.

The disclosed medical treatment system integrates high levels of automation and intelligence, capable of automatically scanning the treatment area, analyzing the data to pinpoint the precise region, and delivering personalized laser treatment plans. Utilizing a multi-axis movement control mode, the system ensures highly targeted treatments with exceptional precision, supported by an intelligent recognition feature. In particular, the medical treatment system combines a hyperspectral camera with AI to offer high levels of automation and intelligence in the treatment process. When the patient's body part is positioned within the treatment apparatus' operational area, the system begins by performing both an image and a 3D spatial scan of the treatment zone. The hyperspectral camera conducts an in-depth spectral analysis, and the resulting images are processed by the system's computer. Specialized algorithms are employed to analyze this data, calculating critical parameters such as the 2D area, position, shape, and size of the targeted region. Subsequently, the controller operates the 3D/6D movement platform, enabling the distance sensor to carry out precise height scanning within the treatment area. This data integration ensures highly accurate 3D spatial positioning and the collection of essential information regarding the treatment zone.

The medical treatment system utilizes cutting-edge narrow-pulse picosecond and/or femtosecond laser technology. With pulse durations in the picosecond (10\hat{ }−12 seconds) or femtosecond range (10\hat{ }−15 seconds), the laser delivers extremely short pulses, high peak power, and wide spectral coverage. These characteristics result in unparalleled precision and minimal thermal or mechanical damage, making it especially suited for achieving exceptional surgical outcomes. For example, the picosecond lasers and femtosecond lasers interact differently with human tissues depending on the target's curvature and depth. These ultrafast laser systems enable precise energy delivery, minimizing thermal diffusion and collateral damage to surrounding tissues. In shallow tissues, the interaction is more focused and localized, while in deeper tissues, the laser penetration relies on the wavelength and energy intensity. Additionally, picosecond and femtosecond lasers exhibit distinct mechanisms for thermal effects, cutting, and drilling in human tissues. In some embodiments, the provided system can also utilize lasers for precise targeting on the surface or inside organs to eliminate harmful cells and tissues. Their ultrafast pulse durations ensure minimal heat accumulation, allowing for efficient material removal and reduced thermal damage during surgical or cosmetic applications.

Moreover, the system is capable of providing tailored scanning plans for various conditions. For instance, the treatment apparatus can offer different laser treatment options designed for specific issues such as scars, pigmentation, tattoos, skin irregularities, signs of aging, skin laxity, moles, and wrinkles. Leveraging data from the intelligent recognition phase, the system analyzes the 2D profile of the treatment area, generating a laser emission zone that is precisely mapped to fit the contours of the target region, even for complex shapes that fall outside of regular geometric configurations. For non-flat areas, such as raised or recessed skin, or for treatments targeting deeper layers within the skin, the system utilizes spatial height data provided by the distance sensor. This information is used to control the Z-axis movement, adjusting the laser's focal depth to accommodate the intricacies of the skin's surface and any underlying treatment needs.

The detailed descriptions of these embodiments illustrate the general principles of the present disclosure. It will be evident to those skilled in the art that these principles can be adapted and applied to various other industrial fields and applications, such as surface or internal material processing, without requiring undue experimentation. Such adaptations and modifications fall within the scope of the disclosed embodiments, as outlined by the teachings provided herein. It should be understood that the terminology and phrasing used herein are intended for illustrative purposes only, not as restrictive limitations. Consequently, the language of this specification should be interpreted by a skilled practitioner in light of the insights and guidance provided.

The embodiments presented above have been described with reference to functional building blocks that outline the implementation of specific functions and their interrelationships. The boundaries of these functional blocks have been defined for convenience in description and are not to be taken as limiting. It is understood that alternate boundaries can be established, provided the required functions and their relationships are still effectively carried out.

The Summary and Abstract sections may present one or more examples of the embodiments as envisioned by the inventor(s), but these are not meant to restrict the disclosure in any way. Thus, the information in these sections should not be viewed as narrowing the scope of the disclosure or the appended claims.

The scope and extent of the present disclosure should be understood to be defined solely by the claims that follow, along with their equivalents, and should not be limited by any of the exemplary embodiments discussed above.

What is claimed is:

1. A system for medical treatment, comprising:
a laser source configured to generate a series of pulsed lasers;
a scanning module configured to provide a series of focused laser spots based on the series of pulsed lasers;
a scanning control module configured to control spatial movement of the scanning module to scan the series of focused laser spots on a treatment area to form a scan pattern; and
a controller configured to control the laser source, the scanning module, and the scanning control module.

2. The system of claim 1, wherein the spatial movement of the scanning module comprises at least one of:
at least three linear movements along three linear directions perpendicular with each other;
at least two linear movements along two linear directions perpendicular with each other, and at least one rotating movement along an angular direction; and
at least a linear movement along a linear direction, a rotating movement along an angular direction, and a radial movement along a radial direction.

3. The system of claim 2, wherein the spatial movement of the scanning module further comprises a roll movement, a yaw movement, and a pitch movement.

4. The system of claim 1, further comprising:
a stage configured to hold a patient including the treatment area;
wherein the controller is further configured to control spatial movement of the stage.

5. The system of claim 4, wherein the spatial movement of the stage comprises at least one of:
at least three linear movements along three linear directions perpendicular with each other;
at least two linear movements along two linear directions perpendicular with each other, and at least one rotating movement along an angular direction; and
at least a linear movement along a linear direction, a rotating movement along an angular direction, and a radial movement along a radial direction.

6. The system of claim 5, wherein the spatial movement of the stage further comprises a roll movement, a yaw movement, and a pitch movement.

7. The system of claim 1, wherein a duration of each of the pulsed lasers is in a range between 1 ns and 1,000 ns, between 1 ps and 1,000 ps, or between 1 fs and 1,000 fs.

8. The system of claim 7, wherein each of the pulsed lasers includes a series of bursts, and each of the focused laser spot is formed by the series of bursts.

9. The system of claim 8, wherein the scanning module is further configured to focus each of the pulsed lasers such that a dimension of each of the focused laser spots is in a range between 0.1 μm and 500 μm.

10. The system of claim 9, wherein the scanning module comprises a scanning galvanometer and at least one laser objective lens.

11. The system of any one of claim 1, wherein the controller is further configured to:
receive detection data indicative of a treatment area; and
provide a control instruction indicative of the scan pattern based on the detection data.

12. The system of claim 11, further comprising an image module configured to, prior to the medical treatment,
detect the treatment area; and
provide the detection data to the controller, wherein the detection data is indicative of at least one of a texture, a shape, a color, and a defect of the treatment area.

13. The system of claim 12, wherein the image module is further configured to, during the medical treatment:
detect the scan pattern; and
provide updated detection data to the controller based on the detection of the scan pattern.

14. The system of claim 13, wherein the scanning control module is further configured to control spatial movement of the scanning module to move the series of focused laser spots on the treatment area to form an updated scan pattern based on the updated detection data.

15. The system of claim 14, wherein the controller is further configured to:
adjust a size and/or a shape of the scan pattern to form the updated scan pattern;
adjust a path of the movement of the focused laser spots to form the updated scan pattern;
process the detection data using a trained AI model to automatically identify and classify a condition type of the treatment area; and
adjust powers applied by the focused laser spots during the medical treatment.

16. The system of claim 15, wherein:
the image module includes a hyperspectral imaging camera configured to capture images of the treatment area across multiple spectral bands, providing diagnostic data.

17. The system of claim 16, further comprising:
a distance measuring module configured to measuring a distance between the scanning module and locations of the treatment area;
wherein the controller is further configured to:
generate a 3D data model of treatment area based on outputs from the image module and the distance measuring module, and
control the scanning module and the scanning control module to adjust at least some of the focused laser spots to form at different depths under a skin of a patient based on the 3D data model of treatment area.

18. The system of claim 1, wherein scanning the series of focused laser spots on the treatment area includes at least one of:
interacting the series of focused laser spots with human tissues at different depths;
generating thermal effects, cutting effects, or perforation effects; and
locating the series of focused laser spots on target harmful cells and tissues on or within organs, enabling non-invasive destruction of undesirable biological structures.

19. A method for medical treatment, comprising:
detecting, by an image module, a treatment area of a patient;
generating, by a laser source, a series of pulsed lasers;
providing, by a scanning module mounted on a 3D or higher-dimensional motion platform, a series of focused laser spots based on the series of pulsed lasers; and
moving the motion platform by a scanning control module, such that the series of focused laser spots on the patient form a scan pattern based on the detected treatment area of the patient.

20. A non-transitory computer-readable medium containing computer-executable instructions that, when executed by a processor, cause the processor to perform a method for medical treatment, the method comprising:
detecting, by an image module, a treatment area of a patient;
generating, by a laser source, a series of pulsed lasers;
providing, by a scanning module mounted on a 3D or higher-dimensional motion platform, a series of focused laser spots based on the series of pulsed lasers; and
moving the motion platform by a scanning control module, such that the series of focused laser spots on the patient form a scan pattern based on the detected treatment area of the patient.
